(19) United States
(12) Patent Application Publication
(10) Pub. No.: US 2011/0262468 A1
(43) Pub. Date: Oct. 27, 2011

(54) Method for Monitoring Vaccine Response Using Single Cell Network Profiling

(75) Inventors: Wendy J. Fantl, San Francisco, CA (US); Diane Longo, Foster City, CA (US); Alessandra Cesano, Redwood City, CA (US)

(73) Assignee: Nodality, Inc., South San Francisco, CA (US)

(21) Appl. No.: 13/091,971

(22) Filed: Apr. 21, 2011

Related U.S. Application Data
(60) Provisional application No. 61/327,347, filed on Apr. 23, 2010.

(51) Int. Cl. A61K 39/00; C12Q 1/02; A61P 37/04

(52) U.S. Cl. 424/184.1; 435/29

(57) ABSTRACT

The present invention provides methods for determining safety and efficacy of a vaccine by monitoring cellular pathways prior to and after vaccine treatment using single cell network profiling

### CROSS-REFERENCE

This application claims the benefit of U.S. Provisional Application No. 61/327,347, filed Apr. 23, 2010, which application is incorporated herein by reference.

### BACKGROUND OF THE INVENTION

A vaccine typically contains an agent that resembles a disease-causing pathogen, and is often made from a weakened or killed form of that pathogen. A vaccine can be prophylactic in that it can prevent or attenuate the effects of a future infection by any naturally occurring pathogen that resembles the agent within the vaccine. Currently approved prophylactic vaccines against viral and bacterial pathogens include but are not limited to: Bordella pertussis, tetanus, diphtheria, influenza, N meningitides serogroup C, hepatitis B, polio, yellow fever, and human papilloma virus. A second class of vaccine can be therapeutic and target a disease that has already manifested. Vaccines intended for therapeutic application are currently in the investigational phase and target conditions including but not limited to cancer, autoimmune disorders, and degenerative disorders, such as Alzheimer's and Parkinson's disease. These vaccines as well as other prophylactic vaccines under development (for example against malaria, human immunodeficiency virus and tuberculosis) must satisfy a number of requirements: 1) safety; 2) production of protective immunity in vaccine recipients; 3) generation of long term immunological memory; and 4) cost effectiveness.

Vaccine development necessarily entails studying cells of the immune system and other relevant cells. Methods must be developed to characterize the heterogeneity of T cell responses (cell-mediated immunity), detection of antibodies and antigen-specific B cells (humoral immunity) and the contribution of innate immunity. One desirable method to monitor vaccine safety and efficacy may be to monitor the immune response before and/or after administration of the vaccine in single cells and/or distinct cell populations simultaneously using at least two activatable elements indicative of immune response activation.

### SUMMARY OF THE INVENTION

One embodiment of the present invention is a method to inform a clinician as to the efficacy of a vaccine-induced immune response in an individual. The method allows for determining a baseline immune functional profile for the individual by analyzing at least two activatable elements in immune system cells comprising cells such as T cells, B cells, myeloid cells, dendritic cells, and NK cells, administering a vaccine to the individual, determining the vaccination immune response for the immune system cells in the individual after the vaccination, correlating the baseline immune functional profile with the vaccination immune response, and inputting the correlations into a database. Another embodiment of the invention is a method to stratify individuals according to their immune response, comprising: determining a baseline immune functional profile for an individual by analyzing at least two activatable elements in immune system cells comprising cells such as T cells, B cells, myeloid cells, and NK cells (optionally simultaneously); correlating the baseline immune functional profile to a database of states of activatable elements; selecting an individual that has an impaired immune response; and optionally providing the individual with immune assistance, which may be adjuvant therapy, one or more cytokines, or another therapy to boost immune function. In another embodiment, the measurement of immune function is selected from the group consisting of: increased: titer, numbers of the following cells: T cells, including cytotoxic T cells, B cells, myeloid cells, NK cells or the activity of any of these cells. These methods may help determine a decision regarding classification, stratification, and/or prediction of an effective vaccine response for the individual.

In some embodiments, the baseline immune profile and/or the vaccination immune response are determined by: i) contacting a first cell from a first cell population from a subject with: (a) at least a first modulator or a fragment thereof, or (b) a presence of no modulator; ii) contacting a second cell from a second cell population from the individual with: (a) at least a second modulator or a fragment thereof, or (b) a presence of no modulator; and iii) determining an activation level of at least one activatable elements in the first cell and the second cell; where the first and second cell population are selected from group consisting of comprising T cells, B cells, myeloid cells, and NK cells.

In one embodiment, the normality or abnormality of an immune response is assessed to understand whether a vaccine may be given to an individual by itself or if some immune assistance should be provided to an individual. Since there is a network of different cell types that contribute to an immune response, analysis of that network will be useful to determine the potency of an individual's immune response. In one embodiment, different profiles using the status of activatable elements, as an example, can be generated for the baseline and/or vaccine treated state. Then, a database can be created with results showing a correlation between a baseline state and/or a vaccine treated state. Specific profiles can be identified that show competent immune response after vaccination and other profiles may be correlated to insufficient immune response for a variety of different reasons. For those that show insufficient immune response, various treatments may be employed to correct the insufficiency. In some embodiments, adjuvant therapy may be provided in conjunction with the vaccination. In others, cytokines, such as the interleukins may be provided during the vaccination process. There will be other mechanisms of assistance in further embodiments. One of the embodiments of the present process allow for the simultaneous analysis of different subsets of cells, different pathways in each of those subsets, and the use of multiple modulators and readouts. The methods allow for inter and intra cellular analysis. Additionally, there is no need for cellular purification.

Some embodiments of the invention consist of the use of biological assays, including but not limited to single cell network profiling (SCNP) to measure the baseline and/or the vaccine induced immune response in single cells within a complex primary sample, for example whole blood or peripheral blood mononuclear cells (PMBCs). Once the vaccine induced immune response from an individual has been characterized, a researcher may be able to design more selective and/or effective vaccines. For example, SCNP may be applied to measure the activated form of a protein, for example, its phosphorylation levels, such as a member of the Signal Transducer and Activator of Transcription (STAT) family, and compare the amount of the activated form, such as the phosphorylated, activated protein to an overall protein level. Protein modifications, including but not limited to phosphorylation, can serve as measurements of both the baseline and vaccine induced immune response by indicating alterations in cellular signaling pathways in response to vaccine treatment. Measurement of such pathway activity can then be utilized for actions such as selecting a particular vaccine antigen, determination of potential unwanted side-effects, adjusting dosing, scheduling, and the like.

In some embodiments determining a baseline immune functional profile and/or a vaccine induced immune response further comprises contacting a sample from at least one individual with at least one modulator, monitoring a baseline and/or a vaccine induced immune response by determining an activation level of at least two activatable elements in different cell subpopulations at the same time. The at least one modulator may be selected from the group consisting of IFNα, IFNγ, IL-2, IL-4, IL-6, IL-10, IL-15, IL-21, IL-27, Baff, TNFα, and the Toll-like Receptor (TLR) ligands Pam3CSK, FSL1, Polyl:C, LPS, Flagellin, Imiquimod, R848, CpG, MDP, PMA, CD40L, TCR, and BCR. The activatable element may be selected from the group consisting of p-Stat1, p-Stat3, p-Stat4, p-Stat5, p-Stat6, p-Akt, p-Erk, p-S6, p38/MAPK, p65/Rel A, TNF-Receptor Associated Factor 6 (TRAF6), MyD88, and NF-κB. The at least one modulator may be a molecule capable of activating a cellular signaling pathway known to regulate or participate in an immune response. The activatable element may be a biomolecule known to initiate or transduce an intracellular signal known to regulate or participate in an immune response.

Measuring the strength of the immune response may further comprise comparing any changes in the activation level of the at least two activatable elements in different cell subpopulations at the same time. Measuring the strength of the immune response may further comprise determining the frequency, lineage, and specificity of T and B cells within a sample collected after vaccine treatment of an individual. Measuring the strength of the immune response may also further comprise determining the frequency, specificity, and titer of any antibodies produced in response to vaccine treatment of an individual. The frequency and specificity of T and B cells and characteristics of the antibody response may be measured using techniques known in the art such as a tetramer assay, a multiplexed bead array assay, ELISA, ELISpot, and the like.

Measuring the baseline and vaccine induced immune response may also comprise monitoring the activation level of at least two activatable elements, at the single cell level of single molecules or a combination of various molecules implicated in transducing immune signaling. Molecules implicated in transducing immune signaling that may be monitored include, but are not limited to: Interleukins (2, 4, 6, 10, 27 as examples) and Interferons (α, β, and γ), Lck, ZAP-70, Fyn, Btk, c-Src, Jak, Fak, Frc, LAT, GSK3, Fos, Jun, Vav, Grb2, PI3K, p-Akt, Nck, PP2A, SHP2, IKKi, IRAK1, IRAK4, TBK, and NFAT. Levels of expression alone, levels of activity alone, or levels of both activity and expression of these molecules may be indicative of an immune response induced by vaccine treatment or predictive of a clinical outcome.

In some embodiments of the invention, the at least two activatable elements comprises biomolecules or motifs within biomolecules that may be modified by epigenetic changes, including but not limited to, methylation, acetylation, ubiquitination, and sumoylation that may regulate levels and/or activity of molecules implicated in transducing immune signaling, including but not limited to p-Stat1, p-Stat3, p-Stat4, p-Stat5, p-Stat6, p-Akt, p-Erk, p-S6, p38/MAPK, p65/Rel A, TNF-Receptor Associated Factor 6 (TRAF6), MyD88, NF-κB, Lck, ZAP-70, Fyn, Btk, c-Src, Jak, Fak, Frc, LAT, GSK3, Fos, Jun, Vav, Grb2, PI3K, p-Akt, Nck, PP2A, SHP2, SOCS proteins, IKKi, IRAK1, IRAK4, TBK and NFAT.

In some embodiments of the invention, the activation level of the at least two activatable elements may be determined using one or more of the following techniques: flow cytometry, cell imaging, mass spectrometry-based flow cytometry, real-time PCR, microarray analysis, thin layer chromatography, or other methods for measuring protein expression and/or modification.

In some embodiments of the invention, the baseline immune functional profile and the vaccine induced immune response may be performed in single cells and various cell subpopulations present in peripheral blood mononuclear cells (PBMCs). For example, this includes CD4+ cells or the CD4− (CD8+) cells with each of their corresponding subpopulations of RA+ (naïve) or RA− (memory) populations. See FIG. 70 of U.S. Ser. No. 61/381,067 which is incorporated by reference. For example including, but not limited to naïve CD4+ T-cells, Thl cells, Th2 cells, Th17 cells, Treg cells, CD8+ cytotoxic T-cells, natural killer (NK) cells, CD19+ B cells, CD20+ and monocytes. Any of the above cell types may be identified and/or isolated based on the presence or absence of at least one cell surface cluster designation (CD) molecule.

In some embodiments, the invention provides methods for the classification, diagnosis, prognosis of disease or prediction of a vaccine response in a subject comprising: a) contacting a first cell from a first cell population from the subject with: (i) at least a first modulator or a fragment thereof, or (ii) a presence of no modulator; b) contacting a second cell from a second cell population from the individual with: (i) at least a second modulator or a fragment thereof, or (ii) a presence of no modulator; c) determining an activation level of at least one activatable element in the first cell and the second cell; and d) classifying, diagnosing, prognosing or predicting of a vaccine response based on the activation level of the at least one activatable element.

In some embodiments, the methods of the invention further comprising creating a response panel for the subject comprising the determined activation levels of the activatable elements. In some embodiments, the first cell population and the second cell population are immune cells. In some embodiments, the first and/or second cell population are selected from the group consisting of CD3+CD4+CD45RA+ naïve helper T cells, CD3+CD4+CD45RA− memory helper, CD3+CD4-CD45RA+naïve cytotoxic T cells, CD3+CD4−CD45RA− memory cytotoxic T cells, CD3+CD8+ cytotoxic T cells, CD3+CD4+Tbet+ TH1 cells, CD3+CD4+GATA3+ TH2 cells, CD3+CD4+CD25+CD127+Foxp3+Tregs cells, CD3+CD4+CCR6+RORyt+TH17 cells, CD3-CD56+ natural killer (NK) cells, CD20+CD19+CD38+B cells, and CD14+CD11b+monocytes.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

Reference will now be made in detail to particularly preferred embodiments of the invention. Examples of the preferred embodiments are illustrated in the following Examples section.

Unless defined otherwise, all technical and scientific terms used herein have the same meaning as is commonly understood by one of skill in the art to which this invention belongs. All patents and publications referred to herein are incorporated by reference in their entirety.

The present invention incorporates information disclosed in other applications and texts. The following publications are hereby incorporated by reference in their entireties: Alberts et al., Molecular Biology of the Cell, 4th Ed., Garland Science, 2002; Michael, Biochemical Pathways, John Wiley and Sons, 1999, Immunobiology, Janeway et al. 7th Ed., Garland, and Leroith and Bondy, Growth Factors and Cytokines in Health and Disease, A Multi Volume Treatise, Volumes 1A and 1B, Growth Factors, 1996; and Immunophenotyping, Chapter 9: Use of Multiparameter Flow Cytometry and Immunophenotyping for the Diagnosis and Classification of Acute Myeloid Leukemia, Stelzer, et al., Wiley, 2000. Abbas A K and Lichtman A H (2003) Cellular and Molecular Immunology (5th ed.) Saunders, Philadelphia.

Patents and applications that are also incorporated by reference in their entirety include U.S. Pat. Nos. 7,381,535, 7,393,656, 7,695,924 and 7,695,926 and U.S. patent application Ser. Nos. 10/193,462; 11/655,785; 11/655,789; 11/655,821; 11/338,957, 12/877,998; 12/784,478; 12/730,170; 12/703,741; 12/687,873; 12/617,438; 12/606,869; 12/713,165; 12/293,081; 12/581,536; 12/776,349; 12/538,643; 12/501,274; 61/079,537 ; 12/501,295; 12/688, 851; 12/471,158 ; 12/910,769; 12/460,029 ; 12/432,239; 12/432,720; 12/229,476, 12/877,998, 61/469812, 61/436,534, PCT/U52011/029845, 61/317,187, and 61/353,155.

Some commercial reagents, protocols, software and instruments that are useful in some embodiments of the present invention are available at the Becton Dickinson Website http://www.bdbiosciences.com/features/products/, and the Beckman Coulter website, http://www.beckmancoulter.com/Default.asp?bhfv=7.

Relevant articles include: High-content single-cell drug screening with phosphospecific flow cytometry, Krutzik et al., Nature Chemical Biology, 23: 132-42, December 2007; Schulz, K. R., et al., Single-cell phospho-protein analysis by flow cytometry, Curr Protoc Immunol, 2007, 78:8 Chapter 8: Units 8.17.1-20, 2007; Krutzik, P. O., et al., Coordinate analysis of murine immune cell surface markers and intracellular phosphoproteins by flow cytometry, J Immunol 2005 Aug 15; 175(4): 2357-65; Krutzik, P.O., et al., Characterization of the murine immunological signaling network with phosphospecific flow cytometry, J Immunol 2005 Aug 15;175(4): 2366-73, 2005; Shulz et al., Current Protocols in Immunology 2007, 78:8.17.1-20; Stelzer et al. Use of Multiparameter Flow Cytometry and Immunophenotyping for the Diagnosis and Classification of Acute Myeloid Leukemia, Immunophenotyping, Wiley, 2000; and Krutzik, P. O. and Nolan, G. P., Intracellular phospho-protein staining techniques for flow cytometry: monitoring single cell signaling events, Cytometry A. 2003 Oct;55(2):61-70, 2005; Krutzik et al, High content single cell drug screening with phosphospecific flow cytometry, Nat Chem Biol. 2008 Feb;4(2):132-42, 2008. Experimental and process protocols and other helpful information can be found at http://proteomics.stanford.edu. The articles and other references cited below are also incorporated by reference in their entireties for all purposes.

### Introduction

In one embodiment, a vaccine is defined as a biological preparation that induces immunity. In another embodiment, that immunity is to a particular condition by stimulating the immune system to recognize an agent associated with the condition as foreign and destroy it. A vaccine may contain one or a plurality of molecules designed to elicit an immune response and long term immunologic memory. In some embodiments, the various molecules within a vaccine may be referred to as antigens or immunogens. In other embodiments, vaccines may be either prophylactic or therapeutic. Prophylactic vaccines, such as the measles vaccine, are designed to prevent fulminant clinical symptoms of a condition while therapeutic vaccines seek to improve or eliminate clinical symptoms by stimulating the immune system to attack an existing condition.

Vaccines are cost effective treatments because they are administered infrequently. In contrast, pharmaceutical therapies must be administered at least weekly for a prolonged and often indefinite time period during treatment of a chronic condition, such as autoimmune disease. A method to allow more rapid assessment of vaccine safety and efficacy would encourage vaccine development and enhance patient compliance.

Development of a vaccine that can be approved for human use is a complex, time consuming process. An effective vaccine must evoke an active immune response by an individual patient's immune system sufficient to stimulate reactive T cells and B cells to respond to the pathological condition targeted by the vaccine and develop immunologic memory to prevent future disease. Development of protective immunity against future infection or immunity sufficient to resolve clinical symptoms of an existing condition does not necessarily follow from a vaccine-induced immune response. Significant biological heterogeneity may exist among patients and this heterogeneity produces diverse, nonuniform immune responses following vaccine treatment. It is important to understand this variability due to age/race/gender. Undesirable vaccines may fail to stimulate a patient's immune system, may not elicit immunologic memory, or may cause harmful side effects. Development and selection of the precise components of a vaccine to maximize an induced immune response and minimize side effects is a major focus of vaccine development.

Vaccines may be monovalent or polyvalent. Monovalent vaccines contain a single antigen designed to evoke an appropriate immune response, and polyvalent vaccines contain at least two antigens designed to evoke an appropriate immune response. The administration of antigens alone can be insufficient to elicit an effective immune response, and adjuvants are included in the vaccine to amplify the immune response. Common adjuvants include various aluminum salts and the organic molecule squalene. Toll-like Receptor (TLR) ligands are also used as adjuvants, and all known TLR ligands act as adjuvants.

TLRs are membrane bound cell surface receptors expressed by leukocytes and mediate immune cell responses to a wide array of antigens. See Bruce Beutler Inferences, questions and possibilities in Toll-like receptor signaling 430 NATURE 257 (2004). At least thirteen different isoforms of the TLR exist. Some studies suggest that TLRs are necessary to generate an inflammatory immune response. Poltorak A. et al. Defective LPS signaling in C3H/HeJ and C57BL/10ScCr mice: mutations in Tlr4 gene 282 SCIENCE 2085 (1998). The binding of a TLR ligand to its cognate receptor initiates a signaling pathway wherein the intracellular adaptor proteins MyD88, TRIF, TRAM, and Tirap are recruited to the TLR. The TLR-adaptor protein complex then activates the cytosolic protein kinases IKKi, IRAK1, IRAK4, and TBK1. These kinases amplify the initial signal provided by the TLR ligand and activate various downstream components of the signaling pathway including, but not limited to the NF-KB transcription factor. The TLR effector kinases ultimately induce an immune response by causing leukocyte proliferation, survival, differentiation, and cytokine production. See Medzhitov R., et al.A human homologue of the Drosophila Toll protein signals activation of adaptive immunity, 388 NATURE 394 (1997). TLR ligands are particularly good adjuvants because they are extremely sensitive to foreign antigens found in vaccines and TLRs are expressed by many cell types, such as: T cells, B cells, macrophages, natural killer cells, epithelial cells, and endothelial cells. The potency of TLR ligands combined with widespread expression of TLRs in many tissue types allows TLRs to elicit a rapid, effective immune response. A method to rapidly evaluate the efficacy of vaccine adjuvants that may include various combinations of TLR ligands would accelerate vaccine development.

In one aspect the invention provides methods to predict a vaccine response. In some embodiments, predicting a vaccine response includes predicting postvaccination titer for each antigen. In some embodiments, predicting a vaccine response includes predicting whether a subject is likely or unlikely to respond to vaccination. In some embodiments, predicting a vaccine response includes predicting a side effect (e.g., a harmful side effect). In some embodiments, predicting a vaccine response includes predicting a response in real time.

A method to predict an effective vaccine induced immune response elicited by a candidate vaccine and monitored in real time would be useful. In the context of the present invention, real time denotes monitoring that may occur as a vaccine induced immune response develops following treatment of an individual with a vaccine. A series of samples may be collected over a predetermined time period to monitor a vaccine induced immune response in real time. In various embodiments of the present invention, single cell network profiling (SCNP) is used to predict patient response to a vaccine and monitor patient response after treatment with at least one vaccine.

### General Methods

One embodiment of the present invention involves the classification, diagnosis, prognosis of disease or outcome after administering a vaccine. Another embodiment of the invention involves monitoring and predicting outcome of disease. In other embodiments, the invention involves the identification of new druggable targets, that can be used alone or in combination with other treatments, including vaccine treatments. The invention allows the selection of patients for specific target therapies, including vaccine therapies. The invention allows for delineation of subpopulations of cells associated with a response to a vaccine, or cells associate with a disease that are differentially susceptible to vaccines, drugs or drug combinations. In another embodiment, the invention provides for the identification of a cell type, that in combination other cell type(s) provide ratiometric or metrics that singly or coordinately allow for surrogate identification of subpopulations of cells associated with a response to a vaccine. In performing these processes, one preferred analysis method involves looking at cell signals and/ or expression markers. One embodiment of cell signal analysis involves the analysis of phosphorylated proteins and the use of flow cytometers in that analysis. In one embodiment, a signal transduction-based classification of vaccines responses can be performed using clustering of phospho-protein patterns or biosignatures. In some embodiments, the present invention provides methods for classification, diagnosis, prognosis of disease and outcome after administering a vaccine by characterizing a plurality of pathways in one or more population of cells. In some embodiments, a treatment is chosen based on the characterization of plurality of pathways in single cells.

In some embodiments, the invention provides methods to classify, diagnose, prognosis of disease or predict outcome after administering a vaccine by: determining an activation level of at least one activatable element in the first cell from a first discrete cell population, where the cell has been optionally contacted with at least a first modulator; and classifying, diagnosing, prognosing of disease or predicting outcome based on the activation level. In some embodiments, the methods further comprise: determining an activation level of at least one activatable element in a second cell from a second discrete cell population, where the cell has been optionally contacted with at least a second modulator; creating a response panel for the individual comprising the determined activation levels of the activatable elements from the first and second cell; and classifying, diagnosing, prognosing of disease or predicting outcome based on the response panel. The first and second modulator can be the same or can be different modulators. Thus, in some embodiments, the invention provides methods for classification, diagnosis, prognosis of disease or prediction of outcome after administering a vaccine in an individual by analyzing a plurality (e.g. two or more) of discrete populations of cells. In some embodiments, the invention provides a method to demarcate discrete populations of cells that correlate with a vaccine response. In some embodiments, the invention provides different discrete populations of cells which analysis in combination allows for the determination of a vaccine response. In some embodiments, the invention provides different discrete populations of cells which analysis in combination allows for the determination of the state of a cellular network. In some embodiments, the invention provides for the determination of a causal association between discrete populations of cells, where the causal association is indicative of the status of a cell network. In another embodiment, the invention provides a method to determine whether one or more cell populations that are part of a cellular network are associated with a vaccine response. A discrete cell population, as used herein, refers to a population of cells in which the majority of cells is of a same cell type or has a same characteristic. In some embodiments, the discrete cell population is an immune cell population (e.g., T cells or B cells).

One method that is useful in the present invention is single cell network profiling (SCNP) as described above. One embodiment of SCNP is an assay that allows simultaneous multiparametric analysis of modulated immune signaling networks at the single cell level in complex tissues, such as whole blood or bone marrow, without need for preanalysis cell isolation. This embodiment may allow monitoring of multiple proteins, or nodes, in multiple cell types that participate in development of an immune response following vaccine treatment. See Todd M. Covey et al., Single Cell Network Profiling (SCNP): Mapping Drug and Target Interactions, Assay Drug Dev. Technol. 2010;8:321-43 hereby incorporated by reference in its entirety.

In some embodiments, SCNP may be used to determine and/or predict the strength of a vaccine induced immune response by determining an immune response in patients prior to vaccine treatment, hereinafter referred to as a baseline immune functional profile. In some embodiments, the invention provides methods to classify, diagnose, prognosis of disease or predict a vaccine response by determining a baseline immune functional profile. That is, in some embodiments, a baseline immune functional profile is used to classify, diagnose, prognosis of disease or predict a vaccine response.

In some embodiments, SCNP may be used to determine and/or predict the strength of a vaccine induced immune response by administering a vaccine to a patient, and then determining a vaccine-induced immune response at a predetermined time or series of time points following vaccine treatment. In some embodiments, the invention provides methods to classify, diagnose, prognosis of disease or predict a vaccine response by determining a vaccine-induced immune response. That is, in some embodiments, a vaccine-induced immune response is used to classify, diagnose, prognosis of disease or predict a vaccine response.

In one embodiment, SCNP may be used to determine and/or predict the strength of a vaccine induced immune response by determining an immune response in patients prior to vaccine treatment, administering a vaccine to a patient, and then determining a vaccine-induced immune response at a predetermined time or series of time points following vaccine treatment. The baseline immune functional profile and the vaccine-induced immune response may be compared, analyzed, and matched to determine the strength of the patient's immune response. The resultant matched data may be stored in a database. This determination of the strength of a patient's immune response using SCNP may reveal the effectiveness and safety of the vaccine with high resolution. For example, an effective vaccine may induce a strong immune response characterized by the simultaneous activation of p-Akt and deactivation of p38/MAPK. This example pattern of concomitant activation-deactivation may then be used for many purposes related to vaccine development such as patient specific monitoring of vaccines for safety and/or efficacy, real time monitoring of a vaccine induced immune response, identifying responsive patients, and stratifying responsive patients based on the strength of each patient's immune response.

At least one patient's baseline immune functional profile may be determined by obtaining a sample from the patient, for example whole peripheral blood, administering at least one modulator to the sample, incubating the sample with the modulator for a predetermined time or a series of time points, and analyzing the immune response of at least one intracellular node by determining the activation level of the activatable element within the intracellular node as described herein. In some embodiments, the activation level of two or more activatable elements is determined. The modulator may include, but is not limited to IFNα, IFNγ, IL-2, IL-4, IL-6, IL-10, IL-15, IL-21, IL-27, Baff, TNFα, and the Toll-like Receptor (TLR) ligands Pam3CSK, FSL1, Polyl:C, LPS, Flagellin, Imiquimod, R848, CpG, MDP, PMA, CD40L, TCR, and BCR. The at least one intracellular node may include, but is not limited to p-Stat1, p-Stat3, p-Stat4, p-Stat5, p-Stat6, p-Akt, p-Erk, p-S6, p38/MAPK, p65/Rel A, TNF-Receptor Associated Factor 6 (TRAF6), MyD88, NF-κB, Lck, ZAP-70, Fyn, Btk, c-Src, Jak, Fak, Frc, LAT, GSK3, Fos, Jun, Vav, Grb2, PI3K, p-Akt, Nck, PP2A, SHP2, SOCS proteins, IKKi, IRAK1, IRAK4, TBK, and NFAT. In some embodiments, the baseline immune functional profile is determined by determining the basal activation level of an activatable element in the sample (e.g., the activation level in response of no modulator). In some embodiments, the baseline immune functional profile is determined by obtaining a sample from the patient, for example whole peripheral blood, exposing a discrete population of cells from the sample to a plurality of modulators (e.g. modulators recited herein) in separate cultures, determining the presence or absence of an increase in activation level of an activatable element in the discrete cell population from each of the separate cultures and classifying the discrete cell population based on the presence or absence of the increase in the activation of the activatable element from each of the separate culture. In some embodiments, the baseline immune functional profile is determined by obtaining a sample from the patient, for example whole peripheral blood, exposing a plurality of discrete populations of cells from the sample to a plurality of modulators (e.g. modulators recited herein) in separate cultures, determining the presence or absence of an increase in activation level of an activatable element in the discrete cell populations from each of the separate cultures and classifying the discrete cell populations based on the presence or absence of the increase in the activation of the activatable element from each of the separate culture. In some embodiments, activation state data is used to characterize multiple pathways in each of the population of cells. The activation state data of the different populations of cells can be used to determine the baseline immune functional profile.

In one embodiment, a patient's vaccine-induced immune response may be determined by administering a vaccine to the patient, collecting a sample after a predetermined time period or a series of time points, administering at least one modulator to the sample, incubating the sample with the modulator for a predetermined time or a series of time points, and analyzing the immune response of at least one intracellular node by determining the activation level of the activatable element within the intracellular node as described herein. In some embodiments, the activation level of two or more activatable elements is determined. The same modulator may be administered and the same intracellular node may be monitored to determine both the patient's baseline immune functional profile and vaccine induced immune response. Analyzing the strength of the immune response of a patient may further comprise determining the frequency of various hematopoietic cells as described herein, quantitating the number of circulating antibodies directed against at least one antigen present in the vaccine, and determining the specificity of the circulating antibodies using techniques known in the art as described herein. In some embodiments, the patient's vaccine-induced immune response is determined by determining the basal activation level of an activatable element in the sample (e.g., the activation level in response of no modulator). In some embodiments, the patient's vaccine-induced immune response is determined by administering a vaccine to the patient, collecting a sample after a predetermined time period or a series of time points, exposing a discrete population of cells from the sample to a plurality of modulators (e.g. modulators recited herein) in separate cultures, determining the presence or absence of an increase in activation level of an activatable element in the discrete cell population from each of the separate cultures and classifying the discrete cell population based on the presence or absence of the increase in the activation of the activatable element from each of the separate culture. In some embodiments, the patient's vaccine-induced immune response is determined by administering a vaccine to the patient, collecting a sample after a predetermined time period or a series of time points, exposing a plurality of discrete populations of cells from the sample to a plurality of modulators (e.g. modulators recited herein) in separate cultures, determining the presence or absence of an increase in activation level of an activatable element in the discrete cell populations from each of the separate cultures and classifying the discrete cell populations based on the presence or absence of the increase in the activation of the activatable element from each of the separate culture. In some embodiments, activation state data is used to characterize multiple pathways in each of the population of cells. The activation state data of the different populations of cells can be used to determine the patient's vaccine-induced immune response.

In some embodiments, the baseline immune functional profile and the vaccine induced immune response may be compared, analyzed, and matched to determine the strength of the immune response. The strength of the immune response may characterize the immune response induced by treatment of patients with at least one vaccine. This characterization may comprise a determination of the activation levels of at least two activatable elements as described herein. Such a determination of the activation levels of the activatable elements may generate a profile of the intracellular signaling pathways that dictate the strength of the immune response. For example, a matched comparison of the patient's baseline immune functional profile and vaccine induced immune response may reveal activation of p-Akt signaling following patient treatment with a vaccine. Such p-Akt activation may be diagnostic of a strong immune response that may effectively protect the patient from disease. The matched comparison of the baseline immune functional profile and the vaccine induced immune response may reveal the effectiveness and safety of the vaccine with high resolution. For example, an effective vaccine may activate the intracellular node p-Akt while simultaneously deactivating the intracellular node p38/MAPK. Each intracellular node monitored may be compared in isolation or together with other monitored activatable elements in any combination.

Multiple baseline immune functional profiles and/or vaccine induced immune responses may be analyzed, compared, and matched to stratify patients based on each patient's differential strength of the immune response. Individual patients or groups of patients may be classified according to an individual or a group's differential strength of the immune response. For example, individual patients or groups of patients may be classified into a group that produces antibodies directed against an antigen present in the administered vaccine and a group that fails to produce antibodies directed against an antigen present in the administered vaccine. The group that produces antibodies directed against an antigen present in the administered vaccine would be deemed to have a high strength of immune response while the group that fails to produce antibodies directed against an antigen present in the administered vaccine would be deemed to have low strength of the immune response. In another example, individual patients or groups of patients may be classified into a group that is at risk of having an adverse side effect to the vaccine.

The baseline immune functional profile and/or the vaccine-induced immune response may be compared, analyzed, and/or matched to determine the strength of the patient's immune response. The resultant matched data may be stored in a database for future comparisons. For example, one profile shown in a baseline immune functional profile may indicate that an individual can produce a strong immune response upon vaccination. This correlation will be inputted into the database. Likewise, a different profile may indicate a weak immune response upon vaccination and this correlation will be inputted into the database. All other profiles of baseline immune functional profile can be correlated to their matching vaccine responses and stored in the database.

In another embodiment of the invention, individuals are tested for their baseline immune functional profile and the results are correlated to profiles in the database and the individual's capacity of producing an immune response is determined. In this method, patients may be stratified in their response and methods for modulating the immune response may be suggested. Example methods include boosting the immune response, such as employing adjuvant therapy or treating with cytokines.

### Samples and Sampling

The methods of the present invention involve analysis of one or more samples from an individual. An individual or a patient is any multicellular organism; in some embodiments, the individual is an animal, for example, a mammal. In some embodiments, the individual is a human.

The sample may be any suitable type that allows for the analysis of different populations of cells. The sample may be any suitable type that allows for the analysis of single populations of cells. Samples may be obtained once or multiple times from an individual. Multiple samples may be obtained from different locations in the individual (e.g., blood samples, bone marrow samples and/or lymph node samples), at different times from the individual (e.g., a series of samples taken to monitor response to treatment with a vaccine or to monitor for return of a pathological condition), or any combination thereof. These and other possible sampling combinations based on the sample type, location and time of sampling allows for the detection of the presence of pre-pathological or pathological cells, the measurement of an evoked immune response and also the monitoring for any condition.

When samples are obtained as a series, e.g., a series of blood samples obtained after treatment with an immunogen of interest to provoke an immune response, the samples may be obtained at fixed times, at intervals determined by the status of the most recent sample or samples or by other characteristics of the individual, or some combination thereof. For example, samples may be obtained at times of approximately 1, 2, 3, or 4 weeks after immunization, at times of approximately 1, 2, 3, 4, 5, 6, 7, 8, 9, 10, or 11 months after immunization, at times of approximately 1, 2, 3, 4, 5, or more than 5 years after immunization, or some combination thereof. It will be appreciated that an interval may not be exact, according to an individual's availability for sampling and the availability of sampling facilities, thus approximate times corresponding to an intended interval scheme are encompassed by the invention. As an example, an individual who has undergone treatment for an autoimmune disease may be sampled (e.g., by blood draw) relatively frequently (e.g., every month or every three months) for the first six months to a year after treatment, then, if circulating antibodies elicited by vaccine treatment are found, less frequently (e.g., at times between one and ten years) thereafter. If, however, any abnormalities, such as a weak immune response or low circulating antibody levels, are found in any of the intervening times, or during the sampling, sampling times may be modified.

Generally, the most easily obtained samples are fluid samples. Fluid samples include normal and pathologic bodily fluids and aspirates of those fluids. Bodily fluids include whole blood, bone marrow aspirate, lymph, and lymph node aspirate. In some embodiments, the sample is a blood sample. In some embodiments, the sample is a bone marrow sample. In some embodiments, the sample is a lymph node sample. In some embodiments, combinations of one or more of a blood, bone marrow, and lymph node sample are used.

In one embodiment, a baseline immune functional profile is analyzed in otherwise normal individuals prior to contact with a vaccine. Their cells are obtained and analyzed for their cell signaling responses by determining a baseline immune functional profile as described herein. In one embodiment, the analysis is performed using a method in which cells are contacted with a modulator and activatable elements are measured to report on the cell signaling pathways prior to the immunization also referred to as treatment with a vaccine. Thereafter, the individual is immunized, cells taken, analyzed for cell signaling function by determining a vaccine induced immune response, the immune response of the individual is tested and then cell signaling function is related to the immunologic response. This analysis creates a database of immunologic and signaling pathway responses which can be stored in an electronic medium and which can be placed into categories for reference against future cell samples. See U.S. Ser. No. 12/538,643 for examples of a database and devices for storing data. For example, a class of responses which correlate signaling and immune response can be established for what could be classified as a normal immunologic response and for other types of responses. Additionally, other categories can be established for immunologic responses with signaling that also relate to combinations of one or more of the following factors such as age, disease state, pathologic or pre-pathologic condition, gender, race, mutational status with respect to particular markers, and the like. Potential responses to a vaccine can be predicted once a cell sample from a second individual is compared to the database.

In one embodiment, a sample may be obtained from an apparently healthy individual during a routine checkup and analyzed so as to provide an assessment of the individual's general health status. In another embodiment, a sample may be taken to screen for commonly occurring side effects both prior to and after treatment with a vaccine. Such screening may encompass testing for a single side effect, a family of related side effects, or a general screening for multiple, unrelated side effects. Screening can be performed weekly, bi-weekly, monthly, bi-monthly, every several months, annually, or in several year intervals and may replace or complement existing screening modalities.

In another embodiment, an individual with a known increased probability of disease occurrence or condition relapse (e.g. if symptoms of autoimmune disease reappear at some time after vaccine treatment) may be monitored regularly to detect the appearance of a particular disease or class of symptoms. An increased probability of disease occurrence or symptom presentation can be based on familial association, age, previous genetic testing results, or occupational, environmental, or therapeutic exposure to disease causing agents. Individuals with increased risk for specific diseases can be monitored regularly for the first signs of an appearance of an abnormal leukocyte population, auto-antibody production, or a decrease in vaccine-elicited circulating antibodies. Monitoring can be performed weekly, bi-weekly, monthly, bi-monthly, every several months, annually, or in several year intervals, or any combination thereof. Monitoring may replace or complement existing screening modalities. Through routine monitoring, early detection of the presence of disease causative or associated cells may result in increased treatment options including treatments with lower toxicity and increased chance of disease control or cure.

In a further embodiment, testing can be performed to confirm or refute the presence of a suspected genetic alteration or cellular physiologic abnormality associated with increased or decreased responsiveness to a particular vaccine. Such methodologies are known in the art. Such testing methodologies include, but are not limited to, techniques such as flow cytometry, cytogenetic analysis, fluorescent in situ histochemistry (FISH), PCR, DNA arrays, and genomic sequencing.

In instances where an individual has a known pre-pathologic or pathologic condition, one or a plurality of cell populations from the appropriate tissue, organ, or organ system can be sampled and analyzed to predict the response of the individual to an available at least one vaccine. In one embodiment, an individual treated with the intent to reduce in number or ablate cells, antibodies, and/or immune modulators (e.g. cytokines and chemokines) that are causative or associated with a pre-pathological or pathological condition can be monitored to assess the decrease in such pathologic condition indicia over time. A reduction in causative or associated cells, antibodies, and/or immune modulators may or may not be associated with the disappearance or lessening of disease symptoms. If the anticipated decrease in pathologic condition indicia does not occur, further treatment with the same or a different treatment regiment may be warranted.

In another embodiment, an individual treated with at least one vaccine to reverse or arrest the progression of a pre-pathological condition can be monitored to assess the reversion rate or percentage of cells arrested at the pre-pathological status point. For example, a patient treated with a vaccine designed to treat the autoimmune disease systemic lupus erthmatosis may be monitored to assess the number of circulating B cells and CD8+T cells producing self-reactive antibodies following treatment with the vaccine. If the anticipated reversion rate is not seen or cells do not arrest at the desired pre-pathological status point further treatment with the same or a different vaccine can be considered.

Individuals may also be monitored for the appearance or increase in cell number, cell type, antibody number, or immune modulator (e.g. cytokines and/or chemokines) associated with a good prognosis. If a beneficial population of cells, antibodies, and/or immune modulators is observed, measures can be taken to further increase their numbers, such as the administration of additional vaccine or adjuvant. Alternatively, individuals may be monitored for the appearance or increase in number of another cell population(s), cell type, antibody number, and/or immune modulator number associated with a poor prognosis. In such a situation, renewed therapy can be considered including continuing, modifying the present vaccine or administering another vaccine.

In these embodiments, one or more samples may be taken from the individual and subjected to a modulator as described herein. In some embodiments, the sample is divided into subsamples that are each subjected to a different modulator. After treatment with the modulator, one or more different populations of cells in the sample or subsample are analyzed to determine their activation level(s). In some embodiments, single cells in one or more different populations are analyzed. Any suitable form of analysis that allows a determination of cell activation level(s) may be used. In some embodiments, the analysis includes the determination of the activation level of an intracellular element, e.g., a protein. In some embodiments, the analysis includes the determination of the activation level of an activatable element, e.g., an intracellular activatable element such as a protein, e.g., a phosphoprotein. Determination of the activation level may be achieved by the use of activation state-specific binding elements, such as antibodies, as described herein. A plurality of activatable elements may be examined in at least one of the different cell populations.

Certain fluid samples can be analyzed in their native state with or without the addition of a diluent or buffer. Alternatively, fluid samples may be further processed to obtain enriched or purified cell populations prior to analysis. Numerous enrichment and purification methodologies for bodily fluids are known in the art. A common method to separate cells from plasma in whole blood is through centrifugation using heparinized tubes. By incorporating a density gradient, further separation of lymphocytes from red blood cells can be achieved. A variety of density gradient media are known in the art including sucrose, dextran, bovine serum albumin (BSA), FICOLL diatrizoate (Pharmacia), FICOLL metrizoate (Nycomed), PERCOLL (Pharmacia), metrizamide, and heavy salts such as cesium chloride. Alternatively, red blood cells can be removed through lysis with an agent such as ammonium chloride prior to centrifugation.

Whole blood can also be applied to filters that are engineered to contain pore sizes that select for the desired cell type or class. For example, rare pathogenic cells or rare hematopoietic cells can be filtered out of diluted, whole blood following lysis of red blood cells by using filters with pore sizes between 5 to 10 μm, as disclosed in U.S. patent application Ser. No. 09/790,673. Alternatively, whole blood can be separated into its constituent cells based on size, shape, deformability, surface receptors, or surface antigens by the use of a microfluidic device as disclosed in U.S. patent application Ser. No. 10/529,453. However, in one embodiment, cell samples to be tested include all the cells required to mount an immunologic response. In another embodiment, the present invention analyzes subsets of T cells, B cells and monocytes. . For example, this includes CD4+ helper T cells or the CD4− (CD8+) cytotoxic T cells with each of their corresponding subpopulations of CD45RA+ (naïve) or CD45RA− (memory) populations. The subsets of T cells, B cells, and monocytes may include, but are not limited to CD3+CD4+CD45RA+ naïve helper T cells, CD3+CD4+CD45RA− memory helper, CD3+CD4−CD45RA+ naïve cytotoxic T cells, CD3+CD4−CD45RA− memory cytotoxic T cells, CD3+CD8+ cytotoxic T cells, CD3+CD4+Tbet+TH1 cells, CD3+CD4+GATA3+TH2 cells, CD3+CD4+CD25+CD127+Foxp3+Tregs cells, CD3+CD4+CCR6+RORγt+TH17 cells, CD3−CD56+ natural killer (NK) cells, CD20+CD19+CD38+B cells, and CD14+CD11b+ monocytes. Additionally, Toll receptor signaling on monocytes can be analyzed.

Cell populations of interest can also be enriched for or isolated from whole blood through positive or negative selection based on the binding of antibodies or other entities that recognize cell surface or cytoplasmic constituents. For example, U.S. Pat. No. 6,190,870 to Schmitz et al. discloses the enrichment of tumor cells from peripheral blood by magnetic sorting of tumor cells that are magnetically labeled with antibodies directed to tissue specific antigens.

See also U.S. Pat. Nos. 7,381,535 and 7,393,656. All of the above patents and applications are incorporated by reference as stated above.

In some embodiments, the cells are cultured post collection in media suitable for revealing the activation level of an activatable element (e.g. RPMI, DMEM) in the presence, or absence, of serum such as fetal bovine serum, bovine serum, human serum, porcine serum, horse serum, goat serum, and the like. When serum is present in the media it could be present at a concentration ranging from 0.0001% to 30%.

### Modulators

The methods and composition of the present invention utilize a modulator. A modulator can be any molecule capable of affecting an immune signaling pathway. In some embodiments, the modulators employed in the present invention may be: IFNα, IFNγ, IL-2, IL-4, IL-6, IL-10, IL-15, IL-21, IL-27, Baff, TNFα, Pam3CSK4, FSL1, Polyl: C, LPS, Flagellin, Imiquimod, R848, CpG, MDP, PMA, CD40L, TCR, and BCR, or any combination of the proceeding.

Modulation can be performed in a variety of environments. In some embodiments, cells are exposed to a modulator immediately after collection. Exposure to a modulator is hereinafter termed modulation. In some embodiments where there is a heterogeneous population of cells, purification of cells is performed after modulation. In some embodiments, whole blood is collected to which a modulator is added. In some embodiments, cells are modulated after processing to isolate single cells or purified fractions of single cells. As an illustrative example, whole blood can be collected and processed for an enriched fraction of lymphocytes, and this enriched fraction is then exposed to a modulator. Modulation can include exposing cells to more than one modulator. For instance, in some embodiments, cells are exposed to at least 2, 3, 4, 5, 6, 7, 8, 9, or 10 modulators. See U.S. Patent Application 61/048,657 which is incorporated by reference.

In some embodiments, cells are cultured post collection in a suitable media before exposure to a modulator. In some embodiments, the media is a growth media. In some embodiments, the growth media is a complex media that may include serum. In some embodiments, the growth media further comprises serum. In some embodiments, the serum is selected from the group consisting of fetal bovine serum, bovine serum, human serum, porcine serum, horse serum, and goat serum. In some embodiments, the serum concentration ranges from 0.0001% to 30%. In some embodiments, the growth media is a chemically defined minimal media and is without serum. In some embodiments, cells are cultured in a differentiating media.

Modulators can act extracellularly or intracellularly. Modulators may produce different effects depending on the concentration and duration of exposure to the single cells or at least one population of cells. An effect of a modulator may further depend on whether the modulators are used in combination or sequentially. Modulators can act directly on an activatable element or indirectly through interaction with one or more intermediary biomolecules. Indirect modulation may include alterations of gene expression wherein the expressed gene product comprises at least two activatable elements or may be a modulator of an activatable element. In some embodiments, the activation level of an activatable element in a cell is determined by contacting the cell with at least 2, 3, 4, 5, 6, 7, 8, 9, or 10 modulators.

In some embodiments, the modulator is an activator. In some embodiments the modulator is an inhibitor. In some embodiments, cells are exposed to one or more modulator. In some embodiments, cells are exposed to at least 2, 3, 4, 5, 6, 7, 8, 9, or 10 modulators. In some embodiments, cells are exposed to at least two modulators, wherein one modulator is an activator and one modulator is an inhibitor. In some embodiments, cells are exposed to at least 2, 3, 4, 5, 6, 7, 8, 9, or 10 modulators, where at least one of the modulators is an inhibitor.

In some embodiments, the baseline immune functional profile and/or the vaccine induced immune response of a population of cells is determined by measuring the activation level of an activatable element when the population of cells is exposed to at least one modulator. The population of cells can be divided into a plurality of samples, and the immune response of the population is determined by measuring the activation level of at least two activatable elements in the samples after the samples have been exposed to at least one modulator. In some embodiments, the baseline immune functional profile and/or the vaccine induced immune response of different populations of cells are determined by measuring the activation level of an activatable element in each population of cells after each of the populations of cells is exposed to a modulator. The different populations of cells can be exposed to the same or different modulators. The immune response of the different populations of cells may also be determined before and after treatment with a vaccine. In some embodiments, a comparison, analysis, and matching of the baseline immune functional profile and the vaccine induced immune response of different cell populations is used to determine the strength of the immune response. The strength of the immune response may be used for the diagnosis, prognosis, and/or selection of a vaccine to be administered to an individual as described herein.

### Vaccines

As used herein, the term vaccine refers to any preparation designed to elicit an immune response. In some embodiments, the preparation may contain an epitope designed to elicit an immune response, such as a protein or protein fragment by itself or attached to a compound such as an adjuvant. The preparation may also contain a killed known pathogen or may contain a live, attenuated form of a known pathogen. The preparation may contain a known pathogen genetically engineered to reduce or eliminate virulence. The preparation may contain synthetic peptides or polypeptides engineered to elicit an immune response. The preparation may contain a DNA expression cassette subcloned into a plasmid vector. The DNA expression cassette may encode one or more peptides and/or polypeptides designed to elicit an immune response. The preparation may further include an adjuvant. Classical adjuvants are well known in the art, and adjuvants within the scope of the present invention further include Toll-like Receptor (TLR) ligands. See Luke A. J. O′Neil & Andrew G. Bowie, The Family of Five: TIR-domain-containing Adaptors in Toll-like Receptor Signaling, 7 Nature Rev. Imm. 353 (2007).

### Determination of the Strength of the Immune Response

In some embodiments, an individual may be treated with a vaccine. At least one sample may be collected from the individual before, after, or before and after treatment with the vaccine. In one embodiment, after treatment with at least one modulator, the sample is analyzed to determine the baseline immune functional profile of at least one cell population. The baseline immune functional profile comprises a profile of intracellular signaling perturbed by the at least one modulator as monitored by determining the activation level of at least two activatable elements as described herein. The population of cells within the sample can be divided into a plurality of samples, and the baseline immune functional profile and/or the vaccine induced immune response of the population is determined by measuring the activation level of at least two activatable elements in the plurality of samples after the samples have been exposed to the at least one modulator.

In some embodiments, the baseline immune functional profile and/or the vaccine induced immune response may be determined by measuring the expression levels and/or activation levels of a plurality of activatable elements at the single cell level. In a preferred embodiment, the plurality of activatable elements comprises modifiable amino acid residues (e.g. serine, threonine, and/or tyrosine residues that may be phosphorylated) or at least one defined structural motif within the polypeptides p-Stat1, p-Stat3, p-Stat4, p-Stat5, p-Stat6, p-Akt, p-Erk, p-S6, p-38, p65/ReIA, TRAF6, MyD88, and NF-κB. Thus, in some embodiments, determining the baseline immune functional profile and/or the vaccine induced immune response involves determining an intracellular signaling profile. In some embodiments, the analysis may be performed in single cells. Any suitable analysis that allows determination of the expression level and/or activation level of an activatable element within single cells, which provides information useful for determining the physiological status of a cell population from whom the sample was taken, may be used. Examples include flow cytometry, immunohistochemistry, immunofluorescent histochemistry with or without confocal microscopy, immunoelectron microscopy, nucleic acid amplification, gene array, protein array, mass spectrometry, patch clamp, gel electrophoresis, 2-dimensional gel electrophoresis, differential display gel electrophoresis, microsphere-based multiplex protein assays, ELISA, Inductively Coupled Plasma Mass Spectrometer (ICP-MS), label-free cellular assays Western immune-blotting, and Far Western blotting. Additional information for the further discrimination between single cells can be obtained by many methods known in the art including the determination of the presence or absence of extracellular and/or intracellular markers, the presence of metabolites, gene expression profiles, DNA sequence analysis, and karyotyping.

### Activatable Elements

The methods and compositions of the invention may be employed to examine and profile the characteristics of the baseline and/or vaccine induced immune response by monitoring the activation level of any activatable element in a cellular pathway, or collections of such activatable elements. Single or multiple distinct pathways may be profiled (sequentially or simultaneously), or subsets of activatable elements within a single pathway or across multiple pathways may be examined sequentially or simultaneously.

As will be appreciated by those in the art, a wide variety of activation events can find use in the present invention to determine the immune response signaling profile. In general, the basic requirement is that the activation results in a change in the activatable protein that is detectable by some indication (termed an “activation state indicator”), preferably by altered binding of a labeled binding element or by changes in detectable biological activities (e.g., the activated state has an enzymatic activity which can be measured and compared to a lack of activity in the non-activated state). Detectable events or moieties may differentiate between two or more activation states accessible to the activatable element. However, in other instances an activatable element may be activated by increased expression such that the increased expression of the activatable element will be measured whether or not there is a differentiating moiety between two or more activation states of the cells.

In some embodiments, the activation state of an individual activatable element may be in the on or off state. As an illustrative example, and without intending to be limited to any theory, an individual phosphorylatable amino acid residue on a protein will either be phosphorylated and transition to the “on” state or it will not be phosphorylated and remain in the “off” state. The terms “on” and “off,” when applied to an activatable element that is a part of a cellular constituent, such as a polypeptide, are used here to describe the state of the activatable element (e.g., phosphorylated is “on” and non-phosphorylated is “off”). The activation state of two activatable elements may not reflect the overall activation state of the cellular constituent of which the activatable element is a part. Typically, a cell possesses a plurality of a particular polypeptide or other constituents with a particular activatable element and this plurality of polypeptides or other constituents usually has some polypeptides or constituents whose individual activatable element is in the on state and other polypeptides or constituents whose individual activatable element is in the off state. Since the activation state of each activatable element may be measured through the use of a binding element that recognizes a specific activation state, only those activatable elements in the specific activation state recognized by the binding element, representing some fraction of the total number of activatable elements accessible to the binding element, will be bound by the binding element to generate a measurable signal. The measurable signal corresponding to the summation of individual detectable activatable elements of a particular type that are activated in a single cell is the “activation level” for that activatable element in that cell.

Activation levels for a particular activatable element may vary among individual cells so that when a plurality of cells is analyzed, the activation levels follow a distribution. The distribution may be a normal distribution, also known as a Gaussian distribution, or it may be of another type. Different populations of cells may have different distributions of activation levels that can serve to distinguish between the populations.

In some embodiments, the basis of determining the activation levels of one or more activatable elements in cells may use the distribution of activation levels for one or more specific activatable elements which will differ among different immune responses. A certain activation level, or more typically a range of activation levels for one or more activatable elements observed in a cell or a population of cells, may indicate that a cell or population of cells exhibits a particular immune response, for example cells that display a range of activation levels for one or more activatable elements may mount a strong immune response following vaccine treatment. Other measurements, such as cellular levels (e.g., expression levels) of biomolecules that may not contain activatable elements, may also be used to determine the strength of the immune response of a cell in addition to activation levels of activatable elements; it will be appreciated that these levels may also follow a distribution, similar to activatable elements. Thus, the activation level or levels of one or more activatable elements, optionally in conjunction with levels of one or more levels of biomolecules that may not contain activatable elements, of one or more cells in a population of cells may be used to determine the strength of the immune response of the cell population.

In some embodiments, the basis for determining the baseline immune functional profile and/or the vaccine induced immune response of a population of cells may use the position of a cell in a contour or density plot. The contour or density plot represents the number of cells that share a characteristic such as the activation level of an activatable protein in response to a modulator or a change in the activation level of an activatable protein in response to a modulator following vaccine treatment. For example, when referring to activation levels of activatable elements in response to one or more modulators, normal individuals and individuals treated with a vaccine may show populations with increased activation levels in response to the one or more modulators. However, the number of cells that have a specific activation level (e.g. specific amount of an activatable element) may differ between normal individuals and individuals treated with a vaccine wherein the vaccine treatment evoked an immune response. Thus, the strength of the immune response of a cell can be determined according to its location within a given region in the contour or density plot.

In addition to activation levels of intracellular activatable elements, expression levels of intracellular or extracellular biomolecules, such as proteins, may be used alone or in combination with activation states of activatable elements to determine the strength of the immune response of a population of cells. Further, additional cellular elements, for example, biomolecules such as RNA, DNA, carbohydrates, metabolites, and the like, may be used in conjunction with activatable states, expression levels or any combination of activatable states and expression levels to determine the strength of the immune response of a single cell or a population of cells.

In some embodiments, other characteristics that affect the activation level of a cellular constituent may also be used to determine the strength of the immune response of a cell. Examples include the translocation of biomolecules or changes in their turnover rates and the formation and disassociation of macromolecular complexes composed of biomolecules. Such macromolecular complexes can include multi-protein complexes composed of a plurality of polypeptides, multi-lipid complexes, homodimers, heterodimers, oligomers, and any combinations thereof.

Additional characteristics may also be used to determine the strength of the immune response of a cell, such as cell volume, cell granularity, nuclear volume, or any other detectable distinguishing characteristic. For example, T cells can be further subdivided based on expression of cell surface markers such as CD3, CD4, CD8, and CD45RA.

In some embodiments, the baseline immune functional profile and/or the vaccine induced immune response of one or more cells is determined by examining and profiling the activation level of one or more activatable elements in a signaling pathway. In some embodiments, the activation levels of one or more activatable elements of a cell from a first population of cells and the activation levels of one or more activatable elements of cell from a second population of cells are correlated with induction of a baseline and/or a vaccine induced immune response. In some embodiments, the first population of cells and the second population of cells are hematopoietic cell populations. For example, this includes CD4+ cells or the CD4− (CD8+) cells with each of their corresponding subpopulations of RA+ (naïve) or RA− (memory) populations. Examples of different populations of hematopoietic cells include, but are not limited to, CD3+CD4+CD45RA+ naïve helper T cells, CD3+CD4+CD45RA− memory helper, CD3+CD4−CD45RA+naïve cytotoxic T cells, CD3+CD4−CD45RA− memory cytotoxic T cells, CD3+CD8+ cytotoxic T cells, CD3+CD4+Tbet+ TH1 cells, CD3+CD4+GATA3+ TH2 cells, CD3+CD4+CD25+CD127+Foxp3+ Tregs cells, CD3+CD4+CCR6+RORγt+ TH17 cells, CD3−CD56+ natural killer (NK) cells, CD20+CD19+CD38+B cells, and CD14+CD11b+ monocytes.

In some embodiments, the activation level of one or more activatable elements in single cells in the sample is determined. Cellular constituents that may include activatable elements include without limitation polypeptides, carbohydrates, lipids, nucleic acids and metabolites. The activatable element may be a portion of the cellular constituent, for example, an amino acid residue in a protein that may undergo phosphorylation, or it may be the cellular constituent itself, for example, a protein that is activated by translocation, conformational change (due to, e.g., change in pH or ion concentration), by proteolytic cleavage, and the like. Upon activation, a change occurs to the activatable element, such as covalent modification of the activatable element (e.g., binding of a molecule or group to the activatable element, such as phosphorylation) or a conformational change. Such changes generally contribute to changes in particular biological, biochemical, or physical properties of the cellular constituent that contains the activatable element. The state of the cellular constituent that contains the activatable element is determined to some degree, though not necessarily completely, by the state of a particular activatable element of the cellular constituent. For example, a polypeptide may have multiple activatable elements, and the particular activation states of these elements may overall determine the activation state of the protein; the state of a single activatable element is not necessarily determinative. Additional factors, such as the binding of other polypeptides, pH, ion concentration, interaction with other cellular constituents, and the like, can also affect the activation state and activation level of the cellular constituent.

In some embodiments, the activation levels of a plurality of intracellular activatable elements in single cells are determined The term “plurality” as used herein refers to two or more. In some embodiments, at least about 2, 3, 4, 5, 6, 7, 8, 9, 10, or more than 10 intracellular activatable elements are determined.

Activation states of activatable elements may result from covalent additions or modifications of biomolecules and include biochemical processes such as glycosylation, phosphorylation, acetylation, methylation, biotinylation, glutamylation, glycylation, hydroxylation, isomerization, prenylation, myristoylation, lipoylation, phosphopantetheinylation, sulfation, ISGylation, nitrosylation, palmitoylation, sumoylation, ubiquitination, neddylation, citrullination, amidation, and disulfide bond formation or disulfide bond reduction. Other possible chemical additions or modifications of biomolecules include the formation of protein carbonyls, direct modifications of protein side chains, such as o-tyrosine, chloro-, nitrotyrosine, and dityrosine, and protein adducts derived from reactions with carbohydrate and lipid derivatives. Other modifications may be non-covalent, such as binding of an allosteric modulator.

In some embodiments, the activatable element is a polypeptide. Examples of polypeptides that may include activatable elements include, but are not limited to kinases, phosphatases, lipid signaling molecules, adaptor/scaffold proteins, cytokines, cytokine regulators, ubiquitination enzymes, adhesion molecules, cytoskeletal/contractile proteins, heterotrimeric G proteins, small molecular weight GTPases, guanine nucleotide exchange factors, GTPase activating proteins, caspases, other polypeptides involved in apoptosis, cell cycle regulators, molecular chaperones, metabolic enzymes, vesicular transport proteins, hydroxylases, isomerases, deacetylases, methylases, demethylases, proteases, ion channels, molecular transporters, transcription factors/DNA binding factors, regulators of transcription, and regulators of translation. Examples of activatable elements, activation states and methods of determining the activation level of activatable elements are described in US Publication Number 20060073474 entitled “Methods and compositions for detecting the activation state of multiple proteins in single cells” and US Publication Number 20050112700 entitled “Methods and compositions for risk stratification” the content of which are incorporated here by reference in their entirety. See also U.S. Pat. Application Nos. 61/048,886, 61/048,920 and Shulz et al, Current Protocols in Immunology 2007, 7:8.17.1-20.

In some embodiments, the polypeptide that may be activated is selected from the group consisting of p-Stat1, p-Stat3, p-Stat4, p-Stat5, p-Stat6, p-Akt, p-Erk, p-S6, p-38, p65/RelA, TRAF6, MyD88, and NF-κB.

In some embodiments of the invention, the methods described herein are employed to determine the activation level of an activatable element, e.g., in an intracellular signaling pathway. Methods and compositions are provided for the determination of the baseline and/or vaccine induced immune response of a cell according to the activation level of an activatable element in an intracellular signaling pathway that may contribute to the strength of the immune response. Methods and compositions are provided for the determination of the immune response of a cell in a first cell population and a cell in a second cell population according to the activation level of an activatable element in a cellular pathway in each cell. The first cell population may be derived from a sample collected before treatment with a vaccine, and the second cell population may be collected after treatment with a vaccine. The second cell population may further comprise a series of samples collected at various times (e.g. a time course) after treatment with a vaccine. The first and second cell populations can be comprised of at least one hematopoietic cell and examples are listed above.

In some embodiments, the determination of the baseline immune functional profile and/or the vaccine induced immune response of cells in different populations according to the activation level of an activatable element, e.g., in a cellular pathway further comprises classifying at least one of the cells as a cell that is correlated with a clinical outcome.

### Gating

In some embodiments of the invention, different gating strategies can be used in order to analyze only relevant subpopulations of cells derived from a sample of mixed population. These gating strategies can be based on the presence of one or more specific surface markers expressed on each cell type. More than one gate may be applied to the sample of mixed population or a subpopulation. For example, B cells may be identified or isolated by gating on CD20+CD19+CD38+ cells. See U.S. Patent Applications 61/085,789, 61/120,320, and 61/079,766, hereby incorporated by reference. See also FIGS. 38 and 59 and accompanying text from U.S. Patent Application 61/440,523 which is incorporated by reference.

### Binding Elements

In some embodiments of the invention, the activation level of an activatable element is determined. One embodiment makes this determination by contacting a plurality of cells from a cell population (e.g. PBMCs) with a binding element that is specific for an activation state of the activatable element. The term “binding element” includes any molecule, e.g., peptide, nucleic acid, small organic molecule which is capable of detecting an activation state of an activatable element over another activation state of the activatable element. Binding elements and labels for binding elements are shown in U.S. Ser. Nos. 61/048,886; 61/048,920 and 61/048,657.

In some embodiments, the binding element is a peptide, polypeptide, oligopeptide or a protein. The peptide, polypeptide, oligopeptide or protein may be made up of naturally occurring amino acids and peptide bonds, or synthetic peptidomimetic structures. Thus “amino acid”, or “peptide residue”, as used herein include both naturally occurring and synthetic amino acids. For example, homo-phenylalanine, citrulline and noreleucine are considered amino acids for the purposes of the invention. The side chains may be in either the (R) or the (S) configuration. In some embodiments, the amino acids are in the S or L-configuration. If amino acids with non-naturally occurring side chains are used, various chemical groups within the amino acid with non-naturally occurring side chains may be substituted, for example to prevent or retard in vivo degradation. Proteins including non-naturally occurring amino acids may be synthesized or in some cases, made recombinantly; see van Hest et al., FEBS Lett 428:(1-2) 68-70 May 22, 1998 and Tang et al., Abstr. Pap Am. Chem. 5218: U138 Part 2 Aug. 22, 1999, both of which are expressly incorporated by reference herein.

Methods of the present invention may be used to detect any activatable element in a sample that is antigenically detectable and antigenically distinguishable from other activatable elements which may be present in the sample. For example, the activation state-specific antibodies of the present invention can be used in the present methods to identify distinct signaling cascades of a subpopulation of complex cell populations; and the ordering of protein activation (e.g., kinase activation) in potential signaling hierarchies. Hence, in some embodiments the expression and phosphorylation of one or more polypeptides are detected and quantified using methods of the present invention. In some embodiments, the expression and phosphorylation of one or more polypeptides that are cellular components of a cellular pathway are detected and quantified using methods of the present invention. As used herein, the term “activation state-specific antibody” or “activation state antibody” or grammatical equivalents thereof, refer to an antibody that specifically binds to a corresponding and specific antigen. Preferably, the corresponding and specific antigen is a specific form of an activatable element. Also preferably, the binding of the activation state-specific antibody is indicative of a specific activation state of a specific activatable element.

In some embodiments, the binding element is an antibody. In some embodiment, the binding element is an activation state-specific antibody, for example an antibody that specifically recognizes the phosphorylated, activated isoform of an intracellular signaling protein such as p-Akt.

The term “antibody” includes full length antibodies and antibody fragments, and may refer to a natural antibody from any organism, an engineered antibody, or an antibody generated recombinantly for experimental, therapeutic, or other purposes. Examples of antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies. The term “antibody” comprises monoclonal and polyclonal antibodies. Antibodies can be antagonists, agonists, neutralizing, inhibitory, or stimulatory. They can be humanized, glycosylated, bound to solid supports, and posses other variations. See U.S. Ser. Nos. 61/048,886; 61/048,920; and 61/048,657.

Activation state specific antibodies can be used to detect kinase activity, however additional means for determining kinase activation are provided by the present invention. For example, substrates that are specifically recognized by protein kinases and phosphorylated thereby are known. Antibodies that specifically bind to such phosphorylated substrates but do not bind to such non-phosphorylated substrates (phospho-specific substrate antibodies) may be used to determine the presence of an activated kinase in a sample.

Activation state specific antibodies rely on the principle that the antigenicity of an activated isoform of an activatable element is distinguishable from the antigenicity of a non-activated isoform of an activatable element or from the antigenicity of an isoform of a different activation state. In some embodiments, an activated isoform of an activatable element possesses an epitope that is absent in a non-activated isoform of an element, or vice versa. In some embodiments, this difference is due to covalent addition of moieties to an element, such as phosphate moieties following phosphorylation, or due to a structural change in an element, as through protein cleavage, or due to an otherwise induced conformational change in an element which causes the element to present the same sequence in an antigenically distinguishable way. In some embodiments, such a conformational change causes an activated isoform of an element to present at least one epitope that is not present in a non-activated isoform, or to not present at least one epitope that is presented by a non-activated isoform of the element. In some embodiments, the epitopes for the distinguishing antibodies are centered proximal to the active site of the element, although as is known in the art, conformational changes in one area of an element may cause alterations in different, possibly distal, areas of the element as well.

Many antibodies, many of which are commercially available (for example, see Cell Signaling Technology, www.cellsignal.com or BD Biosciences, www.bdbiosciences.com) have been produced which specifically bind to the phosphorylated isoform of a protein but do not specifically bind to a non-phosphorylated isoform of a protein. Many such antibodies have been produced for the study of signal transducing proteins which are reversibly phosphorylated. Particularly, many such antibodies have been produced which specifically bind to phosphorylated, activated isoforms of protein. Examples of proteins that can be analyzed with the methods described herein include, but are not limited to, p-Stat1, p-Stat3, p-Stat4, p-Stat5, p-Stat6, p-Akt, p-Erk, p-S6, p38/MAPK, p65/Rel A, TNF-Receptor Associated Factor 6 (TRAF6), MyD88, NF-κB, Lck, ZAP-70, Fyn, Btk, c-Src, Jak, Fak, Frc, LAT, GSK3, Fos, Jun, Vav, Grb2, PI3K, p-Akt, Nck, PP2A, SHP2, SOCS proteins and NFAT.

In some embodiments, an epitope-recognizing fragment of an activation state antibody rather than the whole antibody is used. In some embodiments, the epitope-recognizing fragment is immobilized. In some embodiments, the antibody light chain that recognizes an epitope is used. A recombinant nucleic acid encoding a light chain gene product that recognizes an epitope may be used to produce such an antibody fragment by recombinant means well known in the art.

In some embodiments, the activation state-specific binding element is a peptide comprising a recognition structure that binds to a target structure on an activatable protein. A variety of recognition structures are well known in the art and can be made using methods known in the art, including by phage display libraries (see e.g., Gururaja et al. Chem. Biol. (2000) 7:515-27; Houimel et al., Eur. J. Immunol (2001) 31:3535-45; Cochran et al. J. Am. Chem. Soc. (2001) 123:625-32; Houimel et al. Int. J. Cancer (2001) 92:748-55, each incorporated herein by reference). Further, fluorophores can be attached to such antibodies for use in the methods of the present invention.

A variety of recognition structures are known in the art (e.g., Cochran et al., J. Am. Chem. Soc. (2001) 123:625-32; Boer et al., Blood (2002) 100:467-73, each expressly incorporated herein by reference)) and can be produced using methods known in the art (see e.g., Boer et al., Blood (2002) 100:467-73; Gualillo et al., Mol. Cell Endocrinol. (2002) 190:83-9, each expressly incorporated herein by reference)), including for example combinatorial chemistry methods for producing recognition structures such as polymers with affinity for a target structure on an activatable protein (see e.g., Barn et al., J. Comb. Chem. (2001) 3:534-41; Ju et al., Biotechnol. (1999) 64:232-9, each expressly incorporated herein by reference). In another embodiment, the activation state-specific antibody is a protein that only binds to an isoform of a specific activatable protein that is phosphorylated and does not bind to the isoform of this activatable protein when it is not phosphorylated or nonphosphorylated. In another embodiment the activation state-specific antibody is a protein that only binds to an isoform of an activatable protein that is intracellular and not extracellular, or vice versa. In a some embodiment, the recognition structure is an anti-laminin single-chain antibody fragment (scFv) (see e.g., Sanz et al., Gene Therapy (2002) 9:1049-53; Tse et al., J. Mol. Biol. (2002) 317:85-94, each expressly incorporated herein by reference).

In some embodiments the binding element is a tetramer. A tetramer is a fluorescently labeled macromolecular complex comprising four MHC complexes covalently linked to a synthetic epitope peptide. Tetramers bind antibodies directed against the synthetic epitope peptide and may be used to identify T cells and B cells that present or produce antibodies specific for the synthetic epitope peptide. In one embodiment the binding element is at least one tetramer designed to detect at least one epitope present in a vaccine. Such at least one tetramer may be used to identify and quantify circulating T cells and B cells of interest including, but not limited to circulating T cells and B cells that present or produce antibodies directed against at least one antigen present in a vaccine.

In some embodiments the binding element is an antibody employed in an MBA assay. The MBA assay measures soluble factors secreted by cells. Discrete bead populations are covalently linked to an antibody specific for one soluble factor, for example a cytokine. Each bead population is also identified by a unique fluorescent signature. After incubation with sample, a detection antibody is added and the amount of analyte bound to each bead population is quantified using flow cytometry. MBA may complement the analytical methods described below and has the additional ability to monitor many analytes simultaneously. See R. Chen et al., Simultaneous Quantification of Six Human Cytokines in a Single Sample Using Microparticle-based Flow Cytometric Technology, 45 Clin. Chem. 1693 (1999). An MBA assay may measure a plurality of circulating molecules, for example cytokines and chemokines, and these molecules' particular identities and relative amounts may be indicative of a vaccine-induced immune response.

Examples of activatable elements, activation states and methods of determining the activation level of activatable elements, in part by using various binding elements, are described in US publication number 20060073474 entitled “Methods and compositions for detecting the activation state of multiple proteins in single cells” and US publication number 20050112700 entitled “Methods and compositions for risk stratification” the content of which are incorporate here by reference.

### Labels

The methods and compositions of the instant invention provide binding elements comprising a label or tag. By label is meant a molecule that can be directly (i.e., a primary label) or indirectly (i.e., a secondary label) detected; for example a label can be visualized and/or measured or otherwise identified so that its presence or absence can be known. Binding elements and labels for binding elements are shown in U.S. Ser. Nos. /048,886; 61/048,920 and 61/048,657.

A compound can be directly or indirectly conjugated to a label which provides a detectable signal, e.g. radioisotopes, fluorescent moieties, enzymes, antibodies, particles such as magnetic particles, chemiluminescers, molecules that can be detected by mass spec, specific binding molecules, and the like. Specific binding molecules include pairs, such as biotin and streptavidin, digoxin and antidigoxin etc. Examples of labels include, but are not limited to, optical fluorescent and chromogenic dyes including labels, label enzymes and radioisotopes. In some embodiments of the invention, these labels may be conjugated to the binding elements.

In some embodiments, one or more binding elements are uniquely labeled. Using the example of two activation state specific antibodies, by “uniquely labeled” is meant that a first activation state antibody recognizing a first activated element comprises a first label, and second activation state antibody recognizing a second activated element comprises a second label, wherein the first and second labels are detectable and distinguishable, making the first antibody and the second antibody uniquely labeled.

In general, labels fall into four classes: a) isotopic labels, which may be radioactive or heavy isotopes; b) magnetic, electrical, thermal labels; c) colored, optical labels including luminescent, phosphorous and fluorescent dyes or moieties; and d) binding partners. Labels can also include enzymes (horseradish peroxidase, etc.) and magnetic particles. In some embodiments, the detection label is a primary label. A primary label is one that can be directly detected, such as a fluorophore.

Labels include optical labels such as fluorescent dyes or moieties termed fluorophores. Fluorophores can be either “small molecule” fluors, or proteinaceous, macromolecule fluors (e.g. green fluorescent proteins and all variants thereof).

In some embodiments, activation state-specific antibodies are labeled with quantum dots as disclosed by Chattopadhyay, P. K. et al. Quantum dot semiconductor nanocrystals for immunophenotyping by polychromatic flow cytometry. Nat. Med. 12, 972-977 (2006). Quantum dot labels are commercially available through Invitrogen, http://probes.invitrogen.com/products/qdot/.

Quantum dot labeled antibodies can be used alone or they can be employed in conjunction with organic fluorochrome-conjugated antibodies to increase the total number of labels available. Additionally, activation state-specific antibodies can be labeled using chelated or caged lanthanides as disclosed by Erkki, J. et al. Lanthanide chelates as new fluorochrome labels for cytochemistry. J. Histochemistry Cytochemistry, 36:1449-1451, 1988, and U.S. Pat. No. 7,018850, entitled Salicylamide-Lanthanide Complexes for Use as Luminescent Markers.

In some embodiments, the activatable elements are labeled with tags suitable for Inductively Coupled Plasma Mass Spectrometer (ICP-MS) as disclosed in Tanner et al. Spectrochimica Acta Part B: Atomic Spectroscopy, 2007 Mar;62(3):188-195.

The methods and composition of the present invention may also make use of label enzymes. A label enzyme is an enzyme that may react in the presence of a label enzyme substrate to produce a detectable product. Suitable label enzymes for use in the present invention include but are not limited to, horseradish peroxidase, alkaline phosphatase and glucose oxidase. Methods for the use of such substrates are well known in the art. The presence of the label enzyme is generally revealed through the enzyme's catalysis of a reaction with a label enzyme substrate, producing an identifiable product. Such products may be opaque, such as the reaction of horseradish peroxidase with tetramethyl benzedine, and may have a variety of colors. Other label enzyme substrates, such as Luminol (available from Pierce Chemical Co.), have been developed that produce fluorescent reaction products. Methods for identifying label enzymes with label enzyme substrates are well known in the art and many commercial kits are available. Examples and methods for the use of various label enzymes are described in Savage et al., Previews 247:6-9 (1998), Young, J. Virol. Methods 24:227-236 (1989), which are each hereby incorporated by reference in their entirety.

Labels may be indirectly detected wherein the label is a partner of a binding pair. A partner of a binding pair is one moiety of a first and a second moiety, wherein the first and the second moiety have a specific binding affinity for each other. Suitable binding pairs for use in the invention include, but are not limited to, antigens/antibodies (for example, digoxigenin/anti-digoxigenin, dinitrophenyl (DNP)/anti-DNP, dansyl/anti-dansyl, Fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Other suitable binding pairs include polypeptides such as the FLAG-peptide (Hopp et al., BioTechnology, 6:1204-1210 (1988)); the KT3 epitope peptide (Martin et al., Science, 255: 192-194 (1992)); tubulin epitope peptide (Skinner et al., J. Biol. Chem., 266:15163-15166 (1991)); and the T7 gene 10 protein peptide tag (Lutz-Freyermuth et al., Proc. Natl. Acad. Sci. USA, 87:6393-6397 (1990)) and the antibodies directed against each thereto. As will be appreciated by those in the art, binding pair partners may be used in applications other than for labeling, as is described herein.

As will be appreciated by those in the art, a partner of one binding pair may also be a partner of another binding pair. For example, an antigen (first moiety) may bind to a first antibody (second moiety) that may, in turn, be an antigen for a second antibody (third moiety). It will be further appreciated that such a circumstance allows indirect binding of a first moiety and a third moiety via an intermediary second moiety that is a binding pair partner to each.

As will be appreciated by those in the art, one moiety of a binding pair may comprise a label, as described above. It will further be appreciated that this binding allows for a second moiety to be indirectly labeled upon the binding of a binding partner further comprising a label. Attaching a label to a second moiety that is a partner of a binding pair, as just described, is referred to as indirect labeling.

### Alternative Activation State Indicators

An alternative activation state indicator useful with the instant invention is one that allows for the detection of an activation level of an activatable element by indicating the result of such activation. For example, phosphorylation of a substrate can be used to detect the activation of the kinase responsible for phosphorylating that substrate. Similarly, cleavage of a substrate can be used as an indicator of the activation of a protease responsible for such cleavage. Methods are well known in the art that allow coupling of such indications to detectable signals, such as the labels and tags described above in connection with binding elements. For example, cleavage of a substrate can result in the removal of a quenching moiety and thus allow for a detectable signal to be produced from a previously quenched label.

### Detection

In practicing the methods of this invention, the detection of the activation status of the one or more activatable elements can be carried out by a person, such as a technician in the laboratory. Alternatively, the detection of the status of the one or more activatable elements can be carried out using automated systems. In either case, the detection of the status of the one or more activatable elements for use according to the methods of this invention is performed according to standard techniques and protocols well-established in the art.

One or more activatable elements can be detected and/or quantified by any method that detects and/or quantitates the presence of the activatable element of interest. Such methods may include radioimmunoassay (RIA) or enzyme linked immunoabsorbance assay (ELISA), immunohistochemistry, immunofluorescent histochemistry with or without confocal microscopy, reversed phase assays, homogeneous enzyme immunoassays, and related non-enzymatic techniques, Western blots, whole cell staining, immunoelectron microscopy, nucleic acid amplification, gene array, protein array, mass spectrometry, patch clamp, 2-dimensional gel electrophoresis, differential display gel electrophoresis, microsphere-based multiplex protein assays, label-free cellular assays and flow cytometry, etc. U.S. Pat. No. 4,568,649 describes ligand detection systems, which employ scintillation counting. These techniques are particularly useful for modified protein parameters. Cell readouts for proteins and other cell determinants can be obtained using fluorescent or otherwise tagged reporter molecules. Flow cytometry methods are useful for measuring intracellular parameters. See U.S. patent Ser. No. 10/898,734 and Shulz et al., Current Protocols in Immunology, 2007, 78:8.17.1-20 which are incorporated by reference in their entireties. Instruments that are useful in flow cytometry are available from Becton Dickinson (LSR II, FACSCantoII as examples) or Beckman Coulter (Gallios for example).

In some embodiments, the present invention provides methods for determining the activation level of an activatable element of a single cell. The methods may comprise analyzing a heterogeneous population of cells by flow cytometry on the basis of the activation level of at least two activatable elements. Binding elements (e.g. activation state-specific antibodies) are used to analyze cells on the basis of activatable element activation level, and can be detected as described below. Alternatively, non-binding elements systems as described above can be used in any system described herein.

When using fluorescent labeled components in the methods and compositions of the present invention, it should be recognized that different types of fluorescent monitoring systems, for example cytometric measurement device systems, can be used to practice the invention. In some embodiments, flow cytometric systems are used or systems dedicated to high throughput screening, e.g. assays performed using 96 well or greater microtiter plates. Methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y. -L., San Diego: Academic Press (1989), pp. 219-243; Turro, N. J., Modern Molecular Photochemistry, Menlo Park: Benjamin/Cummings Publishing Col, Inc. (1978), pp. 296-361.

Fluorescence in a sample can be measured using a fluorimeter. In general, excitation radiation, from an excitation source having a first wavelength, passes through excitation optics. The excitation optics cause the excitation radiation to excite the sample. In response, fluorescent proteins in the sample emit radiation that has a second wavelength distinct from the excitation wavelength. Collection optics then collect the emission from the sample. The fluorimeter device can include a temperature controller to maintain the sample at a specific temperature while it is being scanned. According to one embodiment, a multi-axis translation stage moves a microtiter plate holding a plurality of samples in order to position different wells to be collected for analysis. The multi-axis translation stage, temperature controller, auto-focusing feature, and electronics associated with imaging and data collection can be managed by an appropriately programmed digital computer. The computer also can transform the data collected during the assay into another format for presentation. In general, known robotic systems and components can be used.

Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8, each expressly incorporated herein by reference) as well as confocal microscopy. Flow cytometry may also be used to detect fluorescence. In general, flow cytometry involves passage of individual cells through the path of a laser beam. Light scattering and excitation of any fluorescent molecules attached to, or found within, the cell is detected to create a readable output, e.g. cell size, cell granularity, or cellular fluorescent intensity.

The detecting, sorting, or isolating step of the methods of the present invention can entail fluorescence-activated cell sorting (FACS) techniques, where FACS is used to select cells from a population containing a particular surface marker, or the selection step can entail the use of magnetically responsive particles as retrievable supports for target cell capture and/or background removal. A variety of FACS systems are known in the art and can be used in the methods of the invention (see e.g., WO99/54494, filed Apr. 16, 1999; U.S. Ser. No. 20010006787, filed Jul. 5, 2001, each expressly incorporated herein by reference).

In some embodiments, a FACS cell sorter (e.g. a FACSVantage™ Cell Sorter, Becton Dickinson Immunocytometry Systems, San Jose, Calif.) is used to sort and collect cells that may used as an experimental sample or as a population of reference cells. In some embodiments, the experimental sample or reference cells are first contacted with fluorescent-labeled binding elements (e.g. antibodies) directed against specific activatable elements. In such an embodiment, the amount of bound binding element on each cell can be measured by passing droplets containing the cells through the cell sorter. By imparting an electromagnetic charge to droplets containing the positive cells, the cells can be separated from other cells. These positively selected cells can then be harvested in sterile collection vessels. These cell-sorting procedures are described in detail, for example, in the FACSVantage™. Training Manual, with particular reference to sections 3-11 to 3-28 and 10-1 to 10-17, which is hereby incorporated by reference in its entirety.

In another embodiment, a population of cells can be sorted using magnetic separation of cells based on the presence of an isoform of an activatable element. In such separation techniques, cells to be positively selected are first contacted with specific binding element (e.g., an antibody or reagent that binds an isoform of an activatable element). The cells are then contacted with retrievable particles (e.g., magnetically responsive particles) that are coupled with a reagent that binds the specific element. The cell-binding element-particle complex can then be physically separated from non-positive or non-labeled cells, for example, using a magnetic field. When using magnetically responsive particles, the positive or labeled cells can be retained in a container using a magnetic field while the negative cells are removed. These and similar separation procedures are described, for example, in the Baxter Immunotherapy Isolex training manual which is hereby incorporated in its entirety.

In some embodiments, methods for the determination of a receptor element activation state profile for a single cell are provided. The receptor elements may be cell surface receptors or intracellular receptors, such as the estrogen receptor. The methods comprise providing a population of cells and analyzing the population of cells by flow cytometry. Cells are analyzed on the basis of the activation level of at least two activatable elements. In some embodiments, a plurality of activatable element activation-state antibodies may be used to simultaneously determine the activation level of a plurality of activatable elements.

In some embodiments, analysis by flow cytometry on the basis of the activation level of at least two activatable elements is combined with a determination of other flow cytometry readable outputs, such as the presence of cell surface markers, cell granularity and cell size to provide a correlation between the activation level of a plurality of activatable elements and other cell qualities measurable by flow cytometry for single cells.

As will be appreciated, the present invention also provides for the ordering of element clustering events during signal transduction. Particularly, the present invention allows the artisan to construct an element clustering and activation hierarchy based on the correlation of levels of clustering and activation levels of a plurality of activatable elements within single cells. Ordering can be accomplished by comparing the activation level of an activatable element within a single cell or cell population with a control (e.g. an unmodulated and/or no vaccine treated single cell or cell population) at a single time point, or by comparing cells at multiple time points to observe subpopulations of activated signaling that arise over time following treatment of a sample with a modulator or a modulator and a vaccine.

In some embodiments, one or more cells are contained in one or more wells of a 96 well plate or other commercially available multiwell plate. In an alternate embodiment, the reaction mixture or cells are in a cytometric measurement device. Other multiwell plates useful in the present invention include, but are not limited to 384 well plates and 1536 well plates. Still other vessels for containing the reaction mixture or cells and useful in the present invention will be apparent to the skilled artisan.

The addition of the components of the assay for detecting the activation level of an activatable element, or modulation of such activation level, may be sequential or in a predetermined order or grouping under conditions appropriate for the activity that is monitored. Such conditions are described herein and known in the art.

In some embodiments, the activation level of an activatable element is measured using Inductively Coupled Plasma Mass Spectrometer (ICP-MS). A binding element that has been labeled with a specific element binds to the activatable. When the cell is introduced into the ICP, it is atomized and ionized. The elemental composition of the cell, including the labeled binding element bound to the activatable element, is measured. The presence and intensity of the signals corresponding to the labels on the binding element indicates the level of the activatable element associated with the cell (Tanner et al. Spectrochimica Acta Part B: Atomic Spectroscopy, 2007 March; 62(3):188-195).

As will be appreciated by one of skill in the art, the instant methods and compositions find use in a variety of other assay formats in addition to flow cytometry analysis. For example, a chip analogous to a DNA chip can be used in the methods of the present invention. Arrayers and methods for spotting nucleic acids on a chip in a prefigured array are known. In addition, protein chips and methods for synthesis are known. These methods and materials may be adapted for the purpose of affixing activation state binding elements to a chip in a prefigured array. In some embodiments, such a chip comprises a plurality of activatable element binding elements, and is used to determine an activation state profile for elements present on the cell surface. See U.S. Pat. No. 5,744,934.

In some embodiments confocal microscopy can be used to detect activation levels of one or more activatable elements within single cells. Confocal microscopy relies on the serial collection of light from spatially filtered individual specimen points, which is then electronically processed to render a magnified image of the specimen derived from a single focal plane. In some embodiments the binding elements used in connection with confocal microscopy are antibodies conjugated to fluorophores, however other binding elements, such as other proteins or nucleic acids are also possible.

In one embodiment, it is useful to see the profiles for normal individuals as shown in U.S. Ser. No. 61/381,067; Filing Date: Sep. 8, 2010, U.S. Ser. No. 61/440,523; Filing Date: Feb. 8, 2011, U.S. Ser. No. 61/469,812; Filing Date: Mar. 31, 2011 for baseline immune function profile data. These applications are hereby incorporated by reference in their entireties.

In some embodiments, the methods and compositions of the instant invention can be used in conjunction with an “In-Cell Western Assay.” In such an assay, cells may be grown in standard tissue culture vessels using standard tissue culture techniques. Once grown to optimum confluency, the growth media is removed and cells are washed and detached from the vessel surface. The cells can then be counted and volumes sufficient to transfer an appropriate number of cells are aliquoted into microwell plates (e.g., Nunc TM 96 Microwell TM plates). All individual wells may be grown to optimum confluency using standard techniques. The experimental wells may be incubated with a modulator, for example, IL-2. After modulation, cells are fixed and stained with labeled antibodies directed against the at least one activation element of interest. After labeling, the plates can be scanned using an imager such as the Odyssey Imager (LiCor, Lincoln Nebr.) using techniques described in the Odyssey Operator's Manual v1.2, which is hereby incorporated in its entirety.

### Analysis

In some embodiments where flow cytometry is used, flow cytometry experiments are performed and the results are expressed as fold changes using graphical tools and analyses, including, but not limited to a heat map or a histogram to facilitate evaluation. One common way of comparing changes in a set of flow cytometry samples is to overlay histograms of one parameter on the same plot. Flow cytometry experiments ideally include a reference sample against which experimental samples are compared. Reference samples can include normal and/or cells associated with a condition (e.g. tumor cells). See also U.S. Ser. Nos. 61/079,537 and 12/501,295 for visualization tools.

There are methods to take the data from the detection apparatus, such as a flow cytometer, and calculate response. Some methods of analysis, also called metrics are: 1) measuring the difference in the log of the median fluorescence value between an unstimulated fluorochrome-antibody stained sample and a sample that has not been treated with a stimulant or stained (log (MFIUnstimulated Stained)—log (MFIGated Unstained)), 2) measuring the difference in the log of the median fluorescence value between a stimulated fluorochrome-antibody stained sample and a sample that has not been treated with a stimulant or stained (log (MFIStimulated Stained)—log(MFIGated Unstained)), 3) Measuring the change between the stimulated fluorochrome-antibody stained sample and the unstimulated fluorochrome-antibody stained sample log (MFIStimulated Stained)—log (MFIUnstimulated Stained), also called “fold change in median fluorescence intensity”, 4) Measuring the percentage of cells in a Quadrant Gate of a contour plot which measures multiple populations in one or more dimensions, 5) measuring MFI of phospho positive population to obtain percentage positivity above the background; and 6) use of multimodality and spread metrics for large sample population and for subpopulation analysis. Other metrics used to analyze data are population frequency metrics measuring the frequency of cells with a described property such as cells positive for cleaved PARP (% PARP+), or cells positive for p-S6 and p-Akt. Similarly, measurements examining the changes in the frequencies of cells may be applied such as the Change in % PARP+which would measure the % PARP+Stimulated Stained−% PARP+Unstimulated Stained. The AUCunstim metric also measures changes in population frequencies measuring the frequency of cells to become positive compared to an unstimulated condition. See U.S. Ser. No. 12/910,769 which is incorporated by reference in its entirety. Other calculations may be used to obtain corrected results. See U.S. Ser. Nos. 61/436,534, 61/317,187, and PCT/US2011/029845 which are incorporated by reference in their entireties.

Once the SCNP data is obtained, the patients are stratified based on nodes that address vaccine response using a variety of metrics. To stratify the patients based on response or non response to a vaccine, a prioritization of the nodes can be made according to statistical significance or their biological relevance.

One method can use a threshold value for response to the vaccine, such as a value for titer to determine response or non response. Thresholds for titer can be up to the 10th, 20th, 30th, 40th, 50th, 60th, 70th, 80th, 90th and 100th percentile of titer distribution. Another method can use a continuous relationship of the vaccine response, such as titer, to SCNP values. Another method can use a mixture of the two methods in which response is graded into more than two endpoints and less than an infinite number of endpoints, such as 4-10 possible categories of vaccine response.

Once a value is obtained it is correlated to a SCNP profile. For example, response to a vaccine may be linked to one or more SCNP nodes if a node value is above or below a certain threshold. Multiple nodes may be used to better correlate SCNP profiles to outcome, such as response/non-response. Models with multiple nodes are analyzed with the above statistics by weighting different nodes during the analysis. Many statistical methods can be used to develop models for classifying, such as logistic regression, random forest, linear regression, Cox regression, and support vector machines, among others. These methods may show that a patient is 100%, 90%, 80%, 790%, or 60%, likely to respond or not respond.

Advances in flow cytometry have enabled the individual cell enumeration of up to thirteen simultaneous parameters (De Rosa et al., 2001) and are moving towards the study of genomic and proteomic data subsets (Krutzik and Nolan, 2003; Perez and Nolan, 2002). Likewise, advances in other techniques (e.g. microarrays) allow for the identification of multiple activatable elements. As the number of parameters, epitopes, and samples have increased, the complexity of experiments and the challenges of data analysis have grown rapidly. An additional layer of data complexity has been added by the development of stimulation panels which enable the study of activatable elements under a growing set of experimental conditions. See Krutzik et al, Nature Chemical Biology February 2008. Methods for the analysis of multiple parameters are well known in the art. See U.S. Ser. Nos. 61/079,579 and 12/501,295 for gating analysis. See also U.S. patent Ser. Nos. 12/910,769 and 12/460,029.

### EXAMPLES

### Example 1

### Single Cell Network Profiles Associated with Vaccine Response in Healthy Donors 65 Years and Older Using Cryopreserved PBMC Samples

We will use single cell network profiling (SCNP) technology to build biological classifiers that predict vaccine response in healthy subjects at least 65 years of age and lacking HBsAg seroreactivity (i.e. anti-HBsAg<1mIU/mL). The primary objective of the example is to build classifiers for antibody hyporesponses of elderly subjects to selected protein-antigen vaccines using cryopreserved PBMC pre-vaccination (baseline) samples.

All subjects in the study will receive 3 vaccines: Tetanus and Diphtheria booster vaccine (Td), Engerix-B Hepatitis B vaccine (HBsV), and Dukoral Traveler's Diarrhea Vaccine (WC/rBS). Tetanus and Diphtheria vaccine (Td—Canadian generic): single intramuscular dose; Engerix-B Hepatitis B vaccine (HBsV): standard three intramuscular dose regimen; and Dukoral Traveler's Diarrhea Vaccine (WC/rBS): standard two p.o. dose regimen. Vaccine response endpoints (antibody titers) will be measured as follows: Titers for anti-HBsAg will be determined at baseline and at four weeks after the second injection. Titers for anti-tetanus and anti-diphtheria toxoids will be determined at baseline and at four weeks after dosing. Titers for anti-rBS will be determined at baseline and at three weeks after the second dose (4 weeks after the first dose).

SCNP analysis is employed to determine the activation states of signaling pathways using the readouts from the nodes below. After analysis, the profiles developed using SCNP are correlated to the antibody titer assays to obtain profiles for vaccine hyporesponders. Statistical models that predict vaccine responses (titers) using SCNP profiles will be developed and the ability of the models to predict actual titers will be assessed.

Single cell network profiling (SCNP) analysis is conducted in a manner similar to that shown in example 1 of U.S. Ser. No. 12/910,769, example 1 of U.S. Ser. No. 12/713,165 or in example 1 of U.S. Ser. No. 61/381,067. The general procedure is as follows. Samples are thawed and the total cell number is determined by performing a cell count on an AcT10 hematology analyzer. The samples are incubated with the modulators listed below, fixed, and permeabilized. Following permeabilization, the samples are incubated with a cocktail of fluorochrome-conjugated antibodies that recognize extracellular lineage markers and intracellular epitopes including phospho-epitopes within intracellular signaling molecules. Cell viability is assessed by measuring the percentage cells negative for amine aqua and cleaved-PARP at time of thaw.

Approximately 200 subjects (1 cryopreserved PBMC sample at baseline for each subject) will be analyzed in the example. Approximately 2.2 million viable cells post-thaw are required for all assay conditions. An automated process may be used including Hamilton Starlet liquid handling instrumentation. Cell Lines will also be included, as assay controls, to ensure the integrity of the results.

Viability marker Amine Aqua (AA) and extracellular lineage and gating markers CD14, CD20, CD3, CD4, and CD45RA will be assayed in every well. In addition, intracellular signaling nodes will be assayed as shown below in Table 1.

TABLE 1

| | Modulation | Gating & Lineage | Antibody Cocktail/ |
|---|---|---|---|
| Modulator | Time | Markers | Intracellular Readout |
| Autofluorescence | 15 Min | AA, CD14, CD20, | NIH-01 (None-AF |
| (AF) | | CD3, CD4, | background) |
| | | CD45RA | |
| Unmodulated | 15 Min | AA, CD14, CD20, | NIH-07 (c-PARP) |
| | | CD3, CD4, | |
| | | CD45RA | |
| Unmodulated | 15 Min | AA, CD14, CD20, | NIH-02 (p-Stat1, p- |
| | | CD3, CD4, | Stat3) |
| | | CD45RA | |
| IFNα | 15 Min | AA, CD14, CD20, | NIH-02 (p-Stat1, p- |
| | | CD3, CD4, | Stat3) |
| | | CD45RA | |
| IL-6 | 15 Min | AA, CD14, CD20, | NIH-02 (p-Stat1, p- |
| | | CD3, CD4, | Stat3) |
| | | CD45RA | |
| IL-27 | 15 Min | AA, CD14, CD20, | NIH-02 (p-Stat1, p- |
| | | CD3, CD4, | Stat3) |
| | | CD45RA | |
| Unmodulated | 15 Min | AA, CD14, CD20, | NIH-03 (p-Stat5, p- |
| | | CD3, CD4, | Stat6) |
| | | CD45RA | |
| IL-4 | 15 Min | AA, CD14, CD20, | NIH-03 (p-Stat5, p- |
| | | CD3, CD4, | Stat6) |
| | | CD45RA | |
| IL-2 | 15 Min | AA, CD14, CD20, | NIH-03 (p-Stat5, p- |
| | | CD3, CD4, | Stat6) |
| | | CD45RA | |
| IFNα | 15 Min | AA, CD14, CD20, | NIH-03 (p-Stat5, p- |
| | | CD3, CD4, | Stat6) |
| | | CD45RA | |
| IL-27 | 15 Min | AA, CD14, CD20, | NIH-03 (p-Stat5, p- |
| | | CD3, CD4, | Stat6) |
| | | CD45RA | |
| Autofluorescence | 15 Min | AA, CD14, CD20, | NIH-01 (None-AF |
| (AF) | | CD3, CD4, | background) |
| | | CD45RA | |
| Unmodulated | 15 Min | AA, CD14, CD20, | NIH-05 (p-NFkB, p- |
| | | CD3, CD4, | Erk) |
| | | CD45RA | |
| CD40L | 15 Min | AA, CD14, CD20, | NIH-05 (p-NFkB, p- |
| | | CD3, CD4, | Erk) |
| | | CD45RA | |
| R848 | 15 Min | AA, CD14, CD20, | NIH-05 (p-NFkB, p- |
| | | CD3, CD4, | Erk) |
| | | CD45RA | |
| Unmodulated | 15 Min | AA, CD14, CD20, | NIH-04 (p-S6, p-Erk) |
| (DMSO) | | CD3, CD4, | |
| | | CD45RA | |
| PMA | 15 Min | AA, CD14, CD20, | NIH-04 (p-S6, p-Erk) |
| | | CD3, CD4, | |
| | | CD45RA | |
| Autofluorescence | 10 Min | AA, CD14, CD20, | NIH-01 (None-AF |
| (AF) | | CD3, CD4, | background) |
| | | CD45RA | |
| Unmodulated | 10 Min | AA, CD14, CD20, | New Cocktail (p- |
| | | CD3, CD4, | Syk/Zap70, p-Erk) |
| | | CD45RA | |
| Anti-IgM | 10 Min | AA, CD14, CD20, | New Cocktail (p- |
| | | CD3, CD4, | Syk/Zap70, p-Erk) |
| | | CD45RA | |

Profiles of network pathways will be developed after SCNP analysis and these will be correlated to post vaccination titers. The titers may be correlated by using a threshold to establish positive or negative vaccination response or they may be correlated based on the level of the response of the vaccination as compared to the strength of the activation of the network pathways.

Metrics to be used in the statistical analysis: ERF (Equivalent Number of Reference Fluorophores); Fold Change: defined as log2(ERFmodulated/ERFbasal); Total Phospho: defined as log2(ERFmodulated/ERFAF); Basal: defined as log2(ERFunmodulated/ERFAF); Uu is the Mann-Whitney U statistic comparing the ERF values of the modulated and unmodulated wells that has been scaled to the unit interval (0,1) for a given donor. Ua is the same as the Uu metric except that the auto-fluorescence well is used as the reference instead of the unmodulated well. Uq75 is a linear rank statistic designed to identify a shift in the upper quartile of the distribution of the ERF values. ERF values at or below the 75th percentile of the combined distribution of modulated and unmodulated cells are assigned a score of 0. The remaining ERF values are assigned values as in the Uu statistic.

There are age related differences in signaling. For example, see FIGS. 68-77 of U.S. Ser. No. 61/381,067. FIG. 76 shows a variety of different nodes that changed relative to younger donors. These nodes may affect vaccination response. As shown in FIG. 76, the modulator/node combinations and the cells they were tested and found to have a differential response than that of younger patients are: IFNγ/p-Stat1 Naive CD4− T cells; IL2/p-Stat6 Naive CD4+T; IL6/p-Stat1 Naive CD4− T cells; IL6/p-Stat3 Naive CD4− T cells; IFNα/p-Stat5 Naive CD4− T cells; IL2/p-Stat5 Naive CD4+ T cells; IL4/p-Stat5 Naive CD4− T cells; IL10/p-Stat3 Naive CD4− T cells; IL2/p-Stat5 Memory CD4+ T cells; IL27/p-Stat5 Naive CD4− T cells; IL27/p-Stat6 Naive CD4− T cells; IFNα/p-Stat1 B cells; IFNα/p-Stat1 Naive CD4− T cells; and IL10/p-Stat1 B cells.

To summarize the age-associated findings from U.S. Ser. No. 61/381,067, the inventors found 14 age-associated nodes (all interleukins or interferons), 9 of which were in the naïve cytotoxic T cell subset. Several associations found in the child T cell subsets are lacking in the parent T cell population. They also saw inverse age-associations for cytokines with opposing biological functions.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method to inform, prior to a vaccine administration, a clinician as to an individual's vaccine-induced immune response, the method comprising:
a) determining a baseline immune functional profile for the individual by:
i) contacting a first cell from a first cell population from said subject with: (i) at least a first modulator or a fragment thereof, or (ii) a presence of no modulator;
ii) contacting a second cell from a second cell population from said individual with: (i) at least a second modulator or a fragment thereof, or (ii) a presence of no modulator;
iii) determining an activation level of at least one activatable elements in said first cell and said second cell; wherein said first and second cell population are immune system cell populations selected from group consisting of T cells, B cells, myeloid cells, and NK cells;
b) administering a vaccine to the individual;
c) determining the vaccination induced immune response after the vaccination;
d) correlating the baseline immune functional profile with the vaccination induced immune response; and
e) inputting the correlations into a database.

2. A method to stratify individuals according to their immune cell signaling profiles, comprising: determining a baseline immune functional profile for an individual by:
i) contacting a first cell from a first cell population from said subject with: (i) at least a first modulator or a fragment thereof, or (ii) a presence of no modulator;
ii) contacting a second cell from a second cell population from said individual with: (i) at least a second modulator or a fragment thereof, or (ii) a presence of no modulator;
iii) determining an activation level of at least one activatable elements in said first cell and said second cell; wherein said first and second cell population are immune system cell populations selected from group consisting of T cells, B cells, myeloid cells, and NK cells;
correlating the baseline immune functional profile to a database of states of activatable elements; selecting an individual that has an impaired immune profile; and optionally providing the individual with immune assistance.

3. A method in accordance with claim 2, in which the immune assistance is adjuvant therapy, one or more cytokines, or another treatment to boost immune function.

4. A method to determine the ability of an individual to mount a response to a vaccine, comprising;
Obtaining a first blood sample from a first individual prior to contact with a vaccine of interest, the first blood sample comprising a mixture of cells capable of generating an immune response;
Determining a baseline immune functional profile for the individual;
Obtaining a second blood sample from the same individual after contact with a vaccine of interest, the second blood sample comprising a mixture of cells capable of generating an immune response;
Determining a vaccine induced immune response for the individual;
Matching the results of the baseline immune functional profile and vaccine induced immune response to create a matched data set; and
Storing the matched data set in a database.

5. A method to determine the ability of an individual to mount a response to a vaccine comprising:
Obtaining a blood sample from an individual prior to contact with a vaccine of interest, the blood sample comprising a mixture of cells capable of generating an immune response; and
Determining a baseline immune functional profile for the individual; and
Comparing the baseline immune functional profile to a matched data set to assess the individual's capacity for generating an immune response.

6. The method of claim 4 or 5 wherein determining a baseline immune functional profile for the individual further comprises contacting a sample from the individual with at least one modulator and monitoring an immune response by determining an activation level of at least two activatable elements.

7. The method of claim 2 wherein the at least one modulator is selected from the group consisting of IFNα, IFNγ, IL-2, IL-4, IL-6, IL-10, IL-15, IL-21, IL-27, Baff, TNFα, and the Toll-like Receptor (TLR) ligandsPam3CSK, FSL1, Polyl:C, LPS, Flagellin, Imiquimod, R848, CpG, MDP, PMA, CD40L, TCR, and BCR.

8. The method of claim 2 wherein the activatable element is selected from the group consisting of Lck, ZAP-70, Fyn, Btk, c-Src, Jak, Fak, Frc, LAT, GSK3, Fos, Jun, Vav, Grb2, PI3K, p-Akt, Nck, PP2A, SHP2, IKKi, IRAK1, IRAK4, TBK, NFAT, p-Stat1, p-Stat3, p-Stat4, p-Stat5, p-Stat6, p-Akt, p-Erk, p-S6, p38/MAPK, p65/Rel A, TNF-Receptor Associated Factor 6 (TRAF6), MyD88, and NF-κB.

9. The method of claim 4 wherein determining a vaccine-induced immune response further comprises contacting a sample from the individual collected after administering a vaccine to the individual with at least one modulator and monitoring an immune response by determining an activation level of at least two activatable elements within at least one intracellular node.

10. The method of claim 2 wherein the sample is whole blood or peripheral blood mononuclear cells.

11. The method of claim 2 wherein the sample comprises cells selected from the group consisting of CD3+CD4+CD45RA+ naïve helper T cells, CD3+CD4+CD45RA− memory helper, CD3+CD4−CD45RA+ naïve cytotoxic T cells, CD3+CD4−CD45RA− memory cytotoxic T cells, CD3+CD8+cytotoxic T cells, CD3+CD4+Tbet+ TH1 cells, CD3+CD4+GATA3+ TH2 cells, CD3+CD4+CD25+CD127+Foxp3+ Tregs cells, CD3+CD4+CCR6+RORγt+ TH17 cells, CD3−CD56+ natural killer (NK) cells, CD20+CD19+CD38+ B cells, and CD14+CD11b+ monocytes.

12. The method of claim 4 wherein determining a vaccine induced immune function comprises measuring antibody titer or immune cells.

13. A method for the classification, diagnosis, prognosis of disease or prediction of a vaccine response in a subject comprising:
a) contacting a first cell from a first cell population from said subject with: (i) at least a first modulator or a fragment thereof, or (ii) a presence of no modulator;
b) contacting a second cell from a second cell population from said individual with: (i) at least a second modulator or a fragment thereof, or (ii) a presence of no modulator;
c) determining an activation level of at least one activatable element in said first cell and said second cell; and
d) classifying, diagnosing, prognosing or predicting of a vaccine response based on said activation level of said at least one activatable element.

14. The method of claim 13 further comprising creating a response panel for said subject comprising said determined activation levels of said activatable elements.

15. The methods of claim 13 wherein said first cell population and said second cell population are immune cells.

16. The method of claim 13 wherein said first cell from said first cell population are selected from the group consisting of CD3+CD4+CD45RA+ naïve helper T cells, CD3+CD4+CD45RA− memory helper, CD3+CD4−CD45RA+ naïve cytotoxic T cells, CD3+CD4−CD45RA− memory cytotoxic T cells, CD3+CD8+ cytotoxic T cells, CD3+CD4+Tbet+ TH1 cells, CD3+CD4+GATA3+TH2 cells, CD3+CD4+CD25+CD127+Foxp3+ Tregs cells, CD3+CD4+CCR6+RORγt+ TH17 cells, CD3−CD56+ natural killer (NK) cells, CD20+CD19+CD38+ B cells, and CD14+CD11b+ monocytes.

17. The method of claim 13, further comprising administering a vaccine to the individual.
